(19) United States
(12) Patent Application Publication
(10) Pub. No.: US 2026/0209202 A1
(43) Pub. Date: Jul. 23, 2026

(54) N-METHYL-4-(QUINOLIN-2-YL)PYRIDIN-2-AMINE COMPOUNDS

(71) Applicants: Abbvie Inc., North Chicago, IL (US); Abbvie Deutschland Gmbh & CO.KG, Wiesbaden (DE)

(72) Inventors: Marc B. SKADDAN, North Chicago, IL (US); Thomas ERHARD, Wiesbaden (DE); Herve GENESTE, Wiesbaden (DE); Wilfried BRAJE, Wiesbaden (DE); Frauke POHLKI, Wiesbaden (DE)

(21) Appl. No.: 19/107,904

(22) PCT Filed: Aug. 31, 2023
(86) PCT No.: PCT/US2023/031664

§ 371 Date: Feb. 28, 2025

(51) Int. Cl. C07D 401/04; A61K 31/4709; A61P 25/00; C07D 401/14

(52) U.S. Cl. CPC C07D 401/04; A61K 31/4709; A61P 25/00; C07D 401/14

(57) ABSTRACT

The present disclosure provides compounds of Formula (I): and pharmaceutically acceptable salts thereof, wherein R¹, R², and R³ are as defined in the specification. The compounds may be radiolabeled compounds and are useful for diagnostic imaging using positron emission tomography (PET). The compounds of the present disclosure may be used, for example, in diagnostic imaging of 4R tau aggregates.

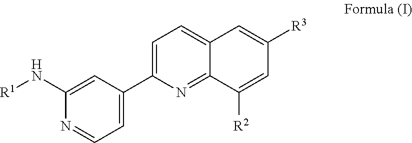

### FIELD

The present disclosure relates to compounds which bind 4-repeat (4R) tau, compositions comprising such compounds, and methods for using these compounds for diagnostic imaging including positron emission tomography (PET).

### BACKGROUND

Tau, a microtubule-associated protein particularly abundant in neurons, regulates microtubule stability and the maintenance of axonal transport. Under physiological conditions, tau binding to microtubules is coordinated by phosphorylation. In pathological conditions, however, increased phosphorylation of tau is associated with a decrease in its binding to microtubules. This in turn results in tau misfolding and self-aggregation, eventually leading to the accumulation of insoluble, paired helical filaments (PHFs) and other filamentous structures.

The pathological aggregation of tau protein is a defining characteristic of the neurodegenerative diseases known as tauopathies, which include Alzheimer's Disease (AD), tangle-only dementia (TD), argyrophilic grain disease (AGD), progressive supranuclear palsy (PSP), corticobasal degeneration (CBD), Pick disease (PiD), and frontotemporal dementia and parkinsonism linked to chromosome 17 (FTDP-17).

The heterogeneity of these disorders is closely related to the wide range of human tau isoforms and post-translational modifications. There are six tau isoforms expressed in human adult brain, which are produced by alternative mRNA splicing of transcripts from the MAPT gene. These isoforms contain either three or four microtubule-binding repeats (3R or 4R tau, respectively), and 0-2 N-terminal inserts (0N, 1N, or 2N tau). Depending on the major tau isoforms appearing in aggregates, tauopathies are usually classified into 3R tauopathies (mainly having 3R tau), 4R tauopathies (mainly having 4R tau) and 3R/4R tauopathies (with approximately an equal ratio of 3R tau and 4R tau). The 4R tauopathies include PSP, CBD and AGD.

The development of effective therapies for treating tauopathies has been hampered by the lack of reliable tools for early diagnosis, staging, and accurately monitoring disease progression. There remains a need to identify an effective means of measuring changes in tau pathology, and in particular there remains a need for compounds capable of reliably imaging tauopathies resulting from aggregations composed primarily of the 4R tau isoform.

### SUMMARY

The present disclosure provides compounds according to Formula (I), or a pharmaceutically acceptable salt thereof,

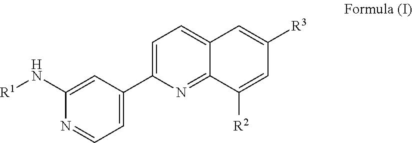

wherein:
R¹ is CH₃ or C(³H)₃;
R² is H, F or ¹⁸F;
R³ is —O—CH₂CH₂—O—CH₃, or

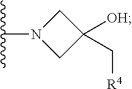

and
R⁴ is F or ¹⁸F.

In embodiments, the compound of Formula (I) is a detectably-labeled compound.

In embodiments, the compound of Formula (I) is an [¹⁸F] radiolabeled compound. In embodiments, the compound of Formula (I) is an [³H] labeled compound.

In embodiments, the present disclosure provides compounds of Formula (I), wherein the compound is selected from the group consisting of:
4-[8-fluoro-6-(2-methoxyethoxy)quinoline-2-yl]-N-methylpyridin-2-amine;
4-[8-(¹⁸F)fluoro-6-(2-methoxyethoxy)quinoline-2-yl]-N-methylpyridin-2-amine;
4-[8-fluoro-6-(2-methoxyethoxy)quinolin-2-yl]-N—(³H₃)methylpyridin-2-amine;
3-(fluoromethyl)-1-{2-[2-(methylamino)pyridin-4-yl]quinolin-6-yl}azetidin-3-ol;
3-[(¹⁸F)fluoromethyl]-1-{2-[2-(methylamino)pyridin-4-yl]quinolin-6-yl}azetidin-3-ol; and
3-(fluoromethyl)-1-(2-{2-[(³H₃)methylamino]pyridin-4-yl}quinolin-6-yl)azetidin-3-ol.

The present disclosure provides a compound according to Compound (I), or a pharmaceutically acceptable salt thereof:

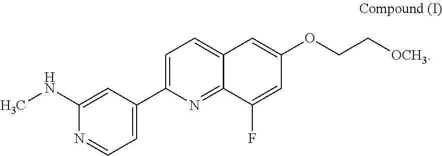 [SMILES: CNc1cc(-c2ccc3cc(OCCOC)cc(F)c3n2)ccn1]

The present disclosure provides an ¹⁸F-labeled compound according to [¹⁸F] Compound (I), or a pharmaceutically acceptable salt thereof:

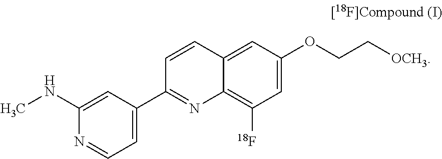 [SMILES: CNc1cc(-c2ccc3cc(OCCOC)cc([18F])c3n2)ccn1]

The present disclosure provides an ³H-labeled compound according to [³H] Compound (I), or a pharmaceutically acceptable salt thereof:

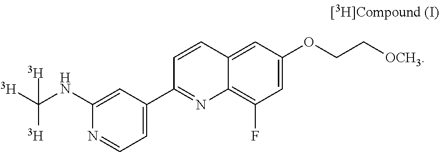 [SMILES: [3H]C([3H])([3H])Nc1cc(-c2ccc3cc(OCCOC)cc(F)c3n2)ccn1]

The present disclosure provides a compound of Compound (II), or a pharmaceutically acceptable salt thereof:

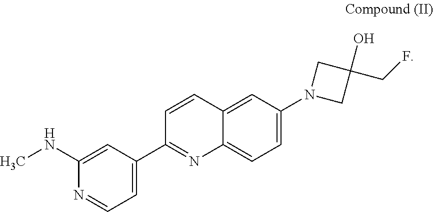 [SMILES: CNc1cc(-c2ccc3cc(N4CC(O)(CF)C4)ccc3n2)ccn1]

The present disclosure provides an ¹⁸F-labeled compound according to [¹⁸F] Compound (II), or a pharmaceutically acceptable salt thereof:

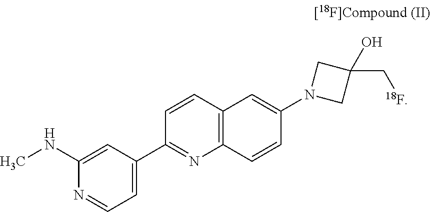

The present disclosure provides an ³H-labeled compound according to [³H] Compound (II), or a pharmaceutically acceptable salt thereof:

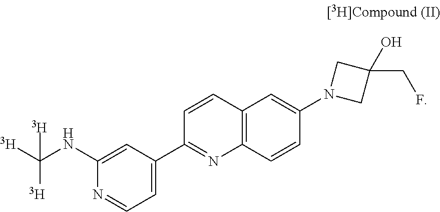 [SMILES: [3H]C([3H])([3H])Nc1cc(-c2ccc3cc(N4CC(O)(CF)C4)ccc3n2)ccn1]

Further provided herein are compositions comprising the compounds of the present disclosure. In embodiments, the present disclosure provides a pharmaceutical composition comprising a compound of Formula (I) or a pharmaceutically acceptable salt thereof, in combination with a pharmaceutically acceptable carrier.

The present disclosure also provides methods for the use of the compounds of the disclosure as imaging agents, for example, in diagnostic imaging of 4R tau aggregates. In embodiments, the compounds may be used for diagnostic imaging using positron emission tomography.

### BRIEF DESCRIPTION OF THE FIGURES

FIG. 1. Autoradiograph showing binding of radiolabeled compounds of the present disclosure in Flash Frozen sections from PSP-affected and healthy brain tissue. The figure shows the distribution of [3H] Compound (I) and [³H] Compound (II) binding to FF sections of the globus pallidus (GP) and putamen (Put) from a representative PSP and a representative normal brain. The figure shows both the binding of the radioligand alone, at 3 nM radioligand alone (total binding) and the binding the radioligand, at 3 nM concentration, in the presence of 10 μM of the corresponding non-radiolabeled compound (non-specific binding).

FIG. 2. Analysis of the binding of radiolabeled compounds of the present disclosure in sections from PSP-affected and healthy brain tissue. Quantitative analysis of [³H] Compound (I) and [³H] Compound (II) binding to the grey matter in sections of FF globus pallidus and putamen from PSP and normal brains. Filled circle, TB (total binding of radioligand) in PSP brain sections; circle, NSB (non-specific binding of radioligand) in PSP brain sections; filled square, TB in PSP brain sections; and square, NSB in normal brain sections.

FIG. 3. Autoradiograph showing binding of radiolabeled compounds of the present disclosure in de-parrafinized sections from PSP-affected and healthy brain tissue. The figure shows distribution of [³H] Compound (I) and [³H] Compound (II) binding to de-paraffinized sections of the FFPE globus pallidus (GP) and putamen (Put) from a representative PSP and a representative normal brain. The figure shows both the binding of the radioligand alone, at 3 nM radioligand alone (total binding) and the binding the radioligand, at 3 nM concentration, in the presence of 10 μM of the corresponding non-radiolabeled compound (non-specific binding). Scale bar, 2 mm.

FIG. 4. Competitive binding curves showing the affinity of radiolabeled compounds of the present disclosure for PSP-affected brain tissue. The figure shows homologous concentration-dependent inhibition of [³H] Compound (I) and [³H] Compound (II) binding to sections of FF globus pallidus and putamen from a representative PSP brain. The upper panel shows the inhibition of [3H] Compound (I) from non-radiolabeled Compound (I); the lower panel shows the inhibition of [³H] Compound (II) from non-radiolabeled Compound (II). ^{sd}K_{D}, constant of dissociation of the radioligand, was determined by means of self-displacement experiment. The ^{sd}K_{D} value indicated in the figure is the mean of values obtained from three different PSP brains.

FIG. 5. Time course of PET tracer entry into the brain of nonhuman primates following intravenous administration. Time Activity Curves (TACs) of [¹⁸F] Compound (I) and [¹⁸F] Compound (II) in nonhuman primates.

FIG. 6. Distribution of PET tracer in the brains of cynomolgus macaques following injection of either [¹⁸F]PET tracers or blocking with unlabeled compound followed by [¹⁸F]PET tracers. Total volume of distribution (V_{T}) vs brain region (baseline and blocking) [¹⁸F] Compound (I) and [¹⁸F] Compound (II) in nonhuman primates.

### DETAILED DESCRIPTION

The present disclosure describes compounds which demonstrate high affinity for 4R tau aggregates. Detectably-labeled compounds of the present disclosure can be employed in the selective detection of disorders associated with tau aggregates such as Progressive Supranuclear Palsy (PSP) and other tauopathies, for example, by using positron emission tomography imaging.

Compounds disclosed herein may contain one or more variable(s) that occur more than one time in any substituent or in the Formulae herein. Definition of a variable on each occurrence is independent of its definition at another occurrence. Further, combinations of substituents are permissible if such combination results in stable compounds. Stable compounds are compounds which can be isolated from a reaction mixture.

### Compounds

In embodiments, the present disclosure provides a compound according to Formula (I), or a pharmaceutically acceptable salt thereof,

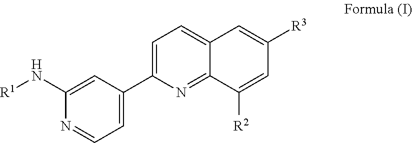

wherein.
R¹ is CH₃ or C(³H)₃;
   R² is H, F or ¹⁸F;
   R³ is —O—CH₂CH₂—O—CH₃, or

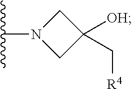

and
R⁴ is F or ¹⁸F.

In embodiments, the present disclosure provides compounds of Formula (I), or a pharmaceutically acceptable salt thereof, wherein R¹ is CH₃ or C(³H)₃, R² is F or ¹⁸F, and R³ is —O—CH₂CH₂—O—CH₃. In embodiments, R¹ is CH₃ and R² is F. In embodiments, R¹ is CH₃ and R² is ¹⁸F. In embodiments, R¹ is C(³H)₃ and R² is F.

In embodiments, the present disclosure provides compounds of Formula (I), or a pharmaceutically acceptable salt thereof, wherein R¹ is CH₃ or C(³H)₃, R² is H, R³ is

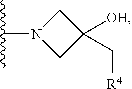

and R⁴ is F or ¹⁸F. In embodiments, R¹ is CH₃ and R⁴ is F. In embodiments, R¹ is CH₃ and R⁴ is ¹⁸F. In embodiments, R¹ is C(³H)₃ and R⁴ is F.

Exemplary compounds of Formula (I) include, for example, the compounds shown in Table 1 below, or a pharmaceutically acceptable salt thereof.

TABLE 1

| EXAMPLE | COMPOUND | STRUCTURE AND NAME |
|---|---|---|
| 1 | Compound (I) | 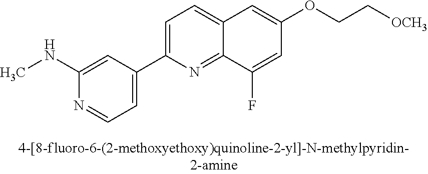 [SMILES: CNc1cc(-c2ccc3cc(OCCOC)cc(F)c3n2)ccn1] |
| 2 | [¹⁸F]Compound (I) | 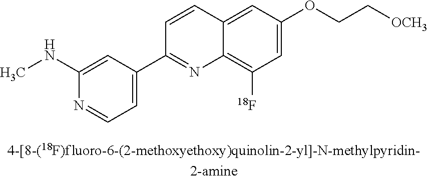 [SMILES: CNc1cc(-c2ccc3cc(OCCOC)cc([18F])c3n2)ccn1] |
| 3 | [³H]Compound (I) | 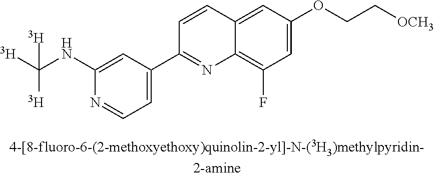 [SMILES: [3H]C([3H])([3H])Nc1cc(-c2ccc3cc(OCCOC)cc(F)c3n2)ccn1] |
| 4 | Compound (II) | 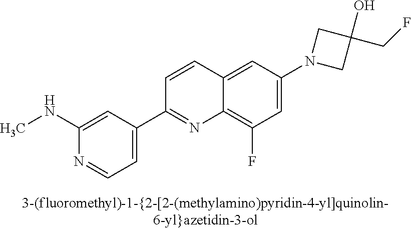 [SMILES: CNc1cc(-c2ccc3cc(N4CC(O)(CF)C4)cc(F)c3n2)ccn1] |
| 5 | [¹⁸F]Compound (II) | 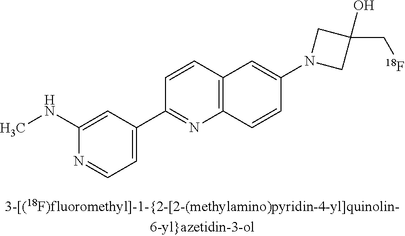 [SMILES: CNc1cc(-c2ccc3cc(N4CC(O)(C[18F])C4)ccc3n2)ccn1] |
| 6 | [³H]Compound (II) | 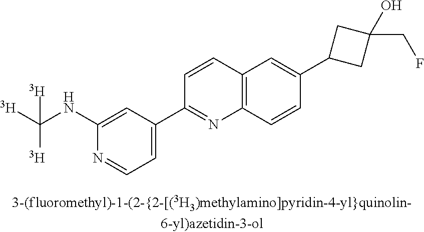 [SMILES: [3H]C([3H])([3H])Nc1cc(-c2ccc3cc(C4CC(O)(CF)C4)ccc3n2)ccn1] |

Compounds of the present disclosure be used in the form of pharmaceutically acceptable salts. The phrase “pharmaceutically acceptable salts” refers to those salts which are, within the scope of sound medical Judgment, suitable for use in contact with the tissues of humans and lower animals without undue toxicity, irritation, allergic response, and the like, and are commensurate with a reasonable benefit/risk ratio.

In embodiments, the present disclosure provides Compound (I), or a pharmaceutically acceptable salt thereof:

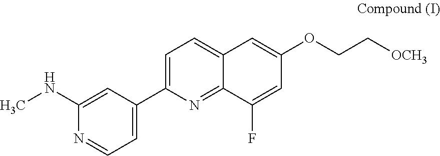 [SMILES: CNc1cc(-c2ccc3cc(OCCOC)cc(F)c3n2)ccn1]

### 4-[8-fluoro-6-(2-methoxyethoxy)quinolin-2-yl]-N-methylpyridin-2-amine

In embodiments, the present disclosure provides an [¹⁸F]-radiolabeled compound according to [¹⁸F] Compound (I), or a pharmaceutically acceptable salt thereof:

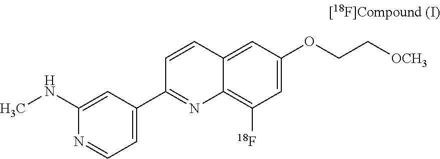 [SMILES: CNc1cc(-c2ccc3cc(OCCOC)cc([18F])c3n2)ccn1]

### 4-[8-(¹⁸F)fluoro-6-(2-methoxyethoxy)quinolin-2-yl]-N-methylpyridin-2-amine

In embodiments, the present disclosure provides a [³H]-radiolabeled compound according to [³H] Compound (I), or a pharmaceutically acceptable salt thereof:

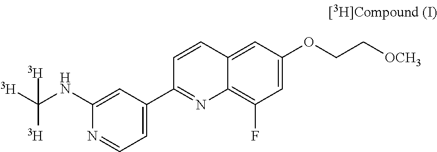 [SMILES: [3H]C([3H])([3H])Nc1cc(-c2ccc3cc(OCCOC)cc(F)c3n2)ccn1]

### 4-[8-fluoro-6-(2-methoxyethoxy)quinolin-2-yl]-N—(³H₃)methylpyridin-2-amine

In embodiments, the present disclosure provides Compound (II), or a pharmaceutically acceptable salt thereof:

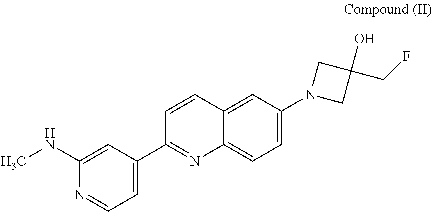 [SMILES: CNc1cc(-c2ccc3cc(N4CC(O)(CF)C4)ccc3n2)ccn1]

### 3-(fluoromethyl)-1-{2-[2-(methylamino)pyridin-4-yl]quinolin-6-yl}azetidin-3-ol

In embodiments, the present disclosure provides an [¹⁸F]-radiolabeled compound, [¹⁸F] Compound (II), or a pharmaceutically acceptable salt thereof:

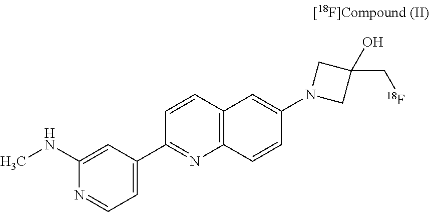 [SMILES: CNc1cc(-c2ccc3cc(N4CC(O)(C[18F])C4)ccc3n2)ccn1]

### 3-[(¹⁸F)fluoromethyl]-1-{2-[2-(methylamino)pyridin-4-yl]quinolin-6-yl}azetidin-3-ol

In embodiments, the present disclosure provides a [³H]-radiolabeled compound, [³H] Compound (II), or a pharmaceutically acceptable salt thereof:

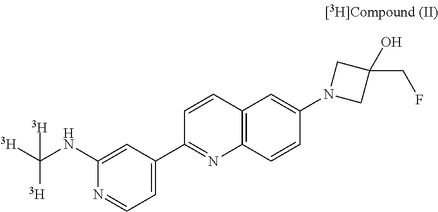 [SMILES: [3H]C([3H])([3H])Nc1cc(-c2ccc3cc(N4CC(O)(CF)C4)ccc3n2)ccn1]

### 3-(fluoromethyl)-1-(2-{2-[(H₃)methylamino]pyridin-4-yl}quinolin-6-yl)azetidin-3-ol

### Pharmaceutical Composition

In embodiments, a compound of the present disclosure may be administered in the form of a pharmaceutical composition. A “pharmaceutical composition” refers to a composition suitable for administration in medical use. Such a composition may comprise a therapeutically or diagnostically effective amount of a compound of Formula (I), or a pharmaceutically acceptable salt thereof, together with a pharmaceutically acceptable carrier.

In embodiments, a pharmaceutical composition is provided comprising a therapeutically or diagnostically effective amount of a compound of Formula (I), or a pharmaceutically acceptable salt thereof, in combination with a pharmaceutically acceptable carrier.

### Method of Use

In embodiments, a radiolabeled compound of the present disclosure may be used as an imaging agent. In embodiments, the compounds of the present disclosure may be used as an in vitro analytical reference or an in vitro screening tool. In embodiments, the compounds of the present disclosure may be used in in vivo diagnostic methods. In embodiments, detectably labeled compounds of the present disclosure may be used for imaging tau aggregates, in particular 4R tau aggregates.

In embodiments, when used for imaging applications, the compounds of the present disclosure may be labeled, or the compound may be unlabeled, and a secondary labeling agent may be used to bind to such molecule. In embodiments, the choice of label may depend on the means of detection. For example, a fluorescent label may be suitable for optical detection. In embodiments, paramagnetic labels and radioisotopic labels can be employed, and may be detected using, for example, positron emission tomography (PET) or single-photon emission computed tomography (SPECT).

In embodiments, the present disclosure provides methods of imaging tau aggregates, comprising administering a compound according to the present disclosure (e.g., a compound according to Formula (I), in particular a detectably labeled compound of Formula (I)) to a patient. In embodiments, the compound is [¹⁸F] Compound (I) or [¹⁸F] Compound (II). In embodiments, the compound is [¹⁸F] Compound (I). In embodiments, the compound is [¹⁸F] Compound (II). In embodiments, the compound is a detectably labeled compound, and the signal stemming from the compound that is specifically bound to the tau aggregates is detected. In embodiments, at least one radiographic image is generated.

In embodiments, the present disclosure provides methods of diagnosing a tauopathy in a patient, the method comprising administering a compound according to the present disclosure (e.g., a compound according to Formula (I), in particular a detectably labeled compound of Formula (I)) to a patient. In embodiments, the compound is [¹⁸F] Compound (I) or [¹⁸F] Compound (II). Diagnosis of a tauopathy in a patient may be achieved by detecting the specific binding of a detectably labeled compound to the 4R tau protein aggregates in the brain of an affected patient. In embodiments, this process may include: (a) contacting the person suspected of a tauopathy with a detectably labeled compound of the present disclosure, such as by injecting the compound into the patient; (b) allowing the detectably labeled compound to bind to 4R tau protein aggregate to form a compound/tau protein aggregate complex (hereinafter “compound/tau protein aggregate complex” will be abbreviated as “compound/protein complex”); (c) detecting the formation of the compound/protein complex, (d) optionally correlating the presence or absence of the compound/protein complex with the presence or absence of 4R tau protein aggregates in the brain of the subject; and (e) optionally comparing the amount of the compound/protein to a normal control value, wherein an increase in the amount of the compound/protein complex compared to a normal control value may indicate that the patient is suffering from or is at risk of developing a 4R tau-associated disorder.

The compound which has bound to the 4R tau protein aggregate can be subsequently detected by an appropriate method. A method of detection is positron emission tomography (PET). PET is a sensitive imaging technique using small quantities of radiolabeled compounds, called “tracers” or “radiotracers”. PET involves introducing the radiopharmaceutical (i.e., a radioisotope attached to a drug) into the body, such as by injection. The labeled compounds preferably are transported, accumulated, and converted in vivo in a similar manner as the corresponding non-radioactively labeled compounds. The labeled compound accumulates in the target tissue and, as it decays, it emits a positron, which promptly combines with a nearby electron resulting in the simultaneous emission of two identifiable gamma rays in opposite directions. The gamma rays are detected by gamma detectors to form a three-dimensional image.

In embodiments, the presence or absence of the compound/protein complex may be correlated with the presence or absence of 4R tau protein aggregates in the brain of a patient. Finally, in embodiments, the amount of the compound/protein complex can be compared to a baseline control value which has been determined in a sample or a specific body part or body area of a healthy subject, wherein an increase in the amount of the compound/protein complex compared to a normal control value may indicate that the patient is suffering from a tau-associated disorder.

In embodiments, diagnosis may be performed by comparing the number, size, and/or intensity of labeled tau conformers, aggregates, and/or neurofibrillary tangles in a sample from the subject or in the subject, to corresponding baseline values. In embodiments, the baseline values can represent the mean levels in a population of non-diseased individuals. In embodiments, baseline values can represent the previous levels determined in the same subject.

In embodiments, the methods of the present disclosure may be employed for diagnosing the presence of a tauopathy. In embodiments, the tauopathy is a four-repeat (4R) tauopathy characterized by cytoplasmic inclusions predominantly composed of tau protein isoforms with four microtubule-binding domains. In embodiments, the 4R tauopathy is progressive supranuclear palsy.

In embodiments, the methods of the present disclosure can also be used to monitor a subject's response to therapy. In embodiments, the presence of 4R tau aggregates is determined prior to the commencement of treatment by administering a detectably labeled compound of the present disclosure. The level of 4R tau aggregates in the subject at this time point is used as a baseline value. The subject is then treated with a therapeutic agent. At various time points during the course of treatment with the therapeutic agent, the detection of tau aggregates is repeated, and the measured values thereafter compared with the baseline values.

The compounds of the present disclosure may also be incorporated into a test kit for detecting a tau protein aggregate. In embodiments, a test kit may comprise a container holding one or more compounds according to the present disclosure. In embodiments, the test kit may comprise instructions for using the compound for binding to a tau protein aggregate, for detecting the formation of a compound/protein complex, and/or for evaluating the presence or absence of the compound/protein complex to correlate with the presence or absence of the tau protein aggregates.

### Examples

In order that the invention described herein may be more fully understood, the following examples are set forth. The synthetic and biological examples described in this application are offered to illustrate the compounds and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

API-ES for atmospheric pressure ionization electrospray; Boc for tert-butoxycarbonyl; (Boc₂O) for di-tert-butyl dicarbonate; t-Bu for tertiary-butyl; tBuXPhos Pd G3 for methanesulfonato(2-di-t-butylphosphino-2′,4′,6′-tri-i-propyl-1,1′-biphenyl)(2′-amino-1,1′-biphenyl-2-yl)palladium(II); DDQ for 2,3-dichloro-5,6-dicyano-1,4-benzoquinone; DMAP for NN-dimethylpyridin-4-amine; DMSO for dimethyl sulfoxide; ESI for electrospray ionization; HIPLC for high performance liquid chromatography; ID for internal diameter; LCMS for liquid chromatography-mass spectrometry; MS for mass spectrum; m/z for mass-to-charge ratio; NMR for nuclear magnetic resonance; ppm for parts per million; PVDF for polyvinylidene difluoride; RuPhos Pd G4 for methanesulfonato(2-dicyclohexylphosphino-2′,6′-di-i-propoxy-1,1′-biphenyl)(2′-methylamino-1,1′-biphenyl-2-yl)palladium(II); SFC for supercritical fluid chromatography; TLC for thin-layer chromatography; and WFI for sterile water for injection.

### Example 1: Preparation of Compound (I), 4-[8-fluoro-6-(2-methoxyethoxy)quinolin-2-yl]-N-methylpyridin-2-amine

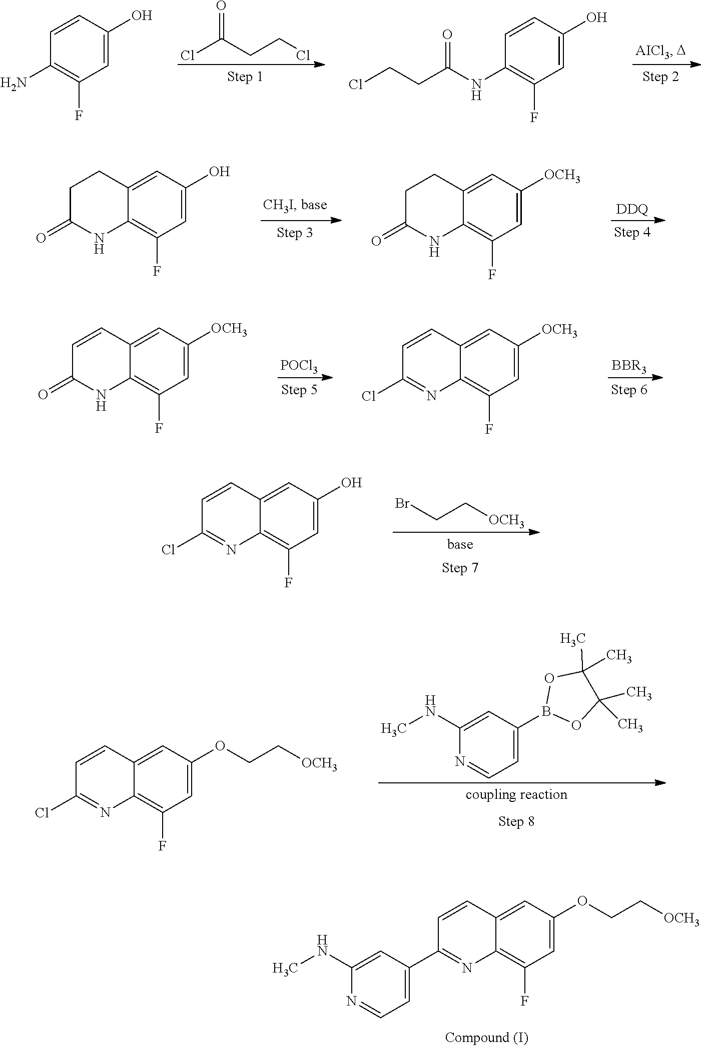

### Example 1-Step 1: 3-chloro-N-(2-fluoro-4-hydroxyphenyl)propanamide

To solution of 3-chloropropanoyl chloride (22.0 g, 173 mmol) in acetone (200 mL) was added 4-amino-3-fluorophenol (20 g, 157 mmol). The mixture was degassed with argon three times. Then the reaction mixture was heated to 60° C. for 3 hours. Nine additional vials were set up and run as described above, and all ten reaction mixtures were then combined. The combined mixture was poured into water (1 L). Ethyl acetate (1 L) was added, and the two phases were separated. The water phase was extracted with ethyl acetate (2×500 mL). The combined organic phases were washed with brine (500 mL), dried over anhydrous Na₂SO₄, filtered, and concentrated. The crude residue was purified by column chromatography on silica gel eluted with petroleum ether/ethyl acetate=2:1 to give the titled compound (220 g, 1.01 mmol, 64% yield). ¹H NMR (400 MHz, DMSO-d₆) δ ppm 9.75 (s, 1H), 9.56 (s, 1H), 7.44 (t, J=9.0 Hz, 1H), 6.64-6.52 (m, 2H), 3.84 (t, J=6.3 Hz, 2H), 2.81 (t, J=6.3 Hz, 2H).

### Example 1-Step 2: 8-fluoro-6-hydroxy-3,4-dihydroquinolin-2(1H)-one

A mixture of aluminum chloride (103 g, 772 mmol) and 3-chloro-N-(2-fluoro-4-hydroxyphenyl)propanamide (28 g, 129 mmol) was stirred at 160° C. for 4 hours. Two additional vials were set up and run as described above, and all three reaction mixtures were then combined. The combined mixture was added to 1 N HCl (800 mL) at 0° C., and the mixture was stirred 10 minutes at 0° C. Ethyl acetate (1 L) was added, and the two phases were separated. The water phase was extracted with ethyl acetate (2×1 L). The combined organic phases were washed with brine (500 mL), dried over anhydrous Na₂SO₄, filtered, and concentrated. The crude residue was purified by precipitation from tert-butyl methyl ether (200 mL). The mixture was filtered, and the filter cake was dried under vacuum to give the titled compound (56.5 g, 312 mmol, 81% yield). ¹H NMR (400 MHz, DMSO-d₆) δ ppm 9.78 (s, 1H), 9.55-9.40 (m, 1H), 6.49-6.39 (m, 2H), 2.86-2.74 (m, 2H), 2.45-2.35 (m, 2H).

### Example 1-Step 3: 8-fluoro-6-methoxy-3,4-dihydroquinolin-2(1H)-one

To solution of 8-fluoro-6-hydroxy-3,4-dihydroquinolin-2(1H)-one (20 g, 88 mmol) and potassium carbonate (18.3 g, 132 mmol) in acetone (30 mL) and N,N-dimethylformamide (30 mL) was added methyl iodide (6.63 mL, 106 mmol) at 0° C. The mixture was stirred at 25° C. for 12 hours. Four additional vials were set up and run as described above, and all five reaction mixtures were then combined. The combined mixture was poured into water (500 mL). Ethyl acetate (500 mL) was added, and the two phases were separated. The water phase was extracted with ethyl acetate (2×250 mL). The combined organic phases were washed with brine (150 mL), dried over anhydrous Na₂SO₄, filtered, and concentrated. The crude product was purified by column chromatography on silica gel eluted with petroleum ether/ethyl acetate=1:1 to give the titled compound (52 g, 266 mmol, 60% yield). ¹H NMR (400 MHz, DMSO-d₆) δ ppm 9.90 (s, 1H), 6.71 (dd, J=2.6, 12.3 Hz, 1H), 6.66 (s, 1H), 3.71 (s, 3H), 2.91-2.84 (m, 2H), 2.43 (dd, J=6.5, 8.2 Hz, 2H).

### Example 1-Step 4: 8-fluoro-6-methoxyquinolin-2(1H)-one

To solution of 8-fluoro-6-methoxy-3,4-dihydroquinolin-2(1H)-one (10 g, 51.2 mmol) in dichloroethane (100 mL) was added 2,3-dichloro-5,6-dicyano-1,4-benzoquinone (34.9 g, 154 mmol). The mixture was stirred at 90° C. for 12 hours. Three additional vials were set up and run as described above, and all four reaction mixtures were then combined. The combined mixture was adjusted to pH=9 with a solution of 1 M aqueous NaOH. Ethyl acetate (500 mL) was added, and the two phases were separated. The water phase was extracted with ethyl acetate (2×500 mL). The combined organic phases were washed with brine (50 mL), dried over anhydrous Na₂SO₄, filtered, and concentrated. The crude product was purified by column chromatography on silica gel eluted with petroleum ether:ethyl acetate=100/1 to 0/1 to give the titled compound (16 g, 83 mmol, 40% yield). ¹H NMR (400 MHz, DMSO-d₆) δ ppm 11.77-11.52 (m, 1H), 7.87 (dd, J=1.5, 9.5 Hz, 1H), 7.16-7.07 (m, 2H), 6.56 (d, J=9.5 Hz, 1H), 3.81-3.78 (m, 3H).

### Example 1-Step 5: 2-chloro-8-fluoro-6-methoxyquinoline

8-Fluoro-6-methoxyquinolin-2(1H)-one (4 g, 20.7 mmol) was dissolved in POCl₃ (16 mL, 172 mmol). The solution was heated at 100° C. for 2 hours. Two additional vials were set up and run as described above, and all three reaction mixtures were then combined. The combined reaction mixture was concentrated under reduced pressure to remove POCl₃. Then the reaction residue was diluted with ethyl acetate (150 mL) and poured into water. Ethyl acetate (150 mL) was added, and the two phases were separated. The water phase was extracted with ethyl acetate (2×100 mL). The combined organic phases were washed with brine (50 mL), dried over anhydrous Na₂SO₄, filtered, and concentrated. The crude product was purified by column chromatography on silica gel eluted with petroleum ether/ethyl acetate=1:1. The crude residue was purified by precipitation from petroleum ether (5 mL) and n-hexane (5 mL). The mixture was filtered. The filter cake was washed with petroleum ether (5 mL) and dried under vacuum to give the titled compound (5.2 g, 22.1 mmol, 35% yield). ¹H NMR (400 MHz, DMSO-d₆) δ ppm 8.39 (dd, J=1.7, 8.6 Hz, 1H), 7.64 (d, J=8.8 Hz, 1H), 7.42 (dd, J=2.4, 12.2 Hz, 1H), 7.34 (d, J=2.0 Hz, 1H), 3.91 (s, 3H); MS (ESI⁺) m/z 211.9 [M+1]⁺.

### Example 1-Step 6: 2-chloro-8-fluoroquinolin-6-ol

2-Chloro-8-fluoro-6-methoxyquinoline (1000 mg, 4.73 mmol) was dissolved in dichloromethane (25 mL) and cooled with an ice-water bath before boron tribromide (14 mL, 14 mmol, 1 molar in dichloromethane) was slowly added under a stream of argon. The reaction mixture was allowed to reach room temperature and stirred overnight. Excess reagent was quenched by slow addition of saturated NaHCO₃ solution followed by dilution with methanol. The crude reaction mixture was poured into 10% NH₄Cl solution and extracted with dichloromethane/methanol (5:1). The combined extracts were dried over MgSO₄ and concentrated in vacuo to yield the titled compound (699 mg, 3.54 mmol, yield 75%) that was used without further purification. MS (ESI⁻) m/z 198 [M+H]⁺.

### Example 1-Step 7: 2-chloro-8-fluoro-6-(2-methoxyethoxy)quinoline

2-Chloro-8-fluoroquinolin-6-ol (231 mg, 1.17 mmol), 1-bromo-2-methoxyethane (0.166 mL, 1.75 mmol, 1.5 equivalents) and K₂CO₃ (485 mg, 3.51 mmol, 3 equivalents) were mixed in N,N-dimethylformamide (5 mL). The reaction mixture was heated to 80° C. for 2 hours to reach complete conversion. The solution of 2-chloro-8-fluoro-6-(2-methoxyethoxy) quinoline (299 mg; 1.17 mmol; yield 100% crude) was used directly in the following Suzuki reaction step (one-pot reaction).

Isolation procedure: The crude reaction mixture was poured into 10% NH₄Cl and extracted with dichloromethane. The combined organic extracts were washed with saturated NaCl, dried over MgSO₄, and concentrated in vacuo to afford the titled compound. MS (ESI⁺) m/z 256 [M+H]⁺.

### Example 1-Step 8: 4-[8-fluoro-6-(2-methoxyethoxy)quinolin-2-yl]-N-methylpyridin-2-amine, Compound (I)

To a crude solution of 2-chloro-8-fluoro-6-(2-methoxyethoxy) quinoline (299 mg; 1.17 mmol) in N,N-dimethylformamide (5 mL) were added N-methyl-4-(4,4,5,5-tetramethyl-1,3,2-dioxaborolan-2-yl)pyridine-2-amine (329 mg, 1.4 mmol; 1.2 equivalents), [1,1′-bis(diphenylphosphino)ferrocene]dichloropalladium(II), complex with dichloromethane (96 mg; 0.12 mmol; 0.1 equivalent) and Cs₂CO₃ (381 mg; 1.17 mmol, 1 equivalent), and the mixture was further diluted with dioxane (5 mL) and water (2 mL). After sparging with argon, the reaction mixture was heated to 80° C. for 1 hour to reach complete conversion. The cooled crude mixture was poured into saturated NaHCO₃ solution and extracted with dichloromethane. The combined organic extracts were washed with saturated NaCl solution, dried over MgSO₄, and concentrated in vacuo. The residue was pre-purified by flash chromatography on silica, eluted with 0-100% ethyl acetate/cyclohexane, followed by SFC purification (Column: VDS 100 Diol, 150×32 mm, 5 μm; Eluents: (A) supercritical CO₂, (B) methanol+30% dichloromethane+0.2% NH₄OH; Isocratic 90/10 (A/B); Flow: 120 mL/minute) to afford the titled compound (219 mg, 0.67 mmol, yield 57%). ¹H NMR (500 MHz, DMSO-d₆) δ ppm 8.41 (dd, J=8.8, 1.5 Hz, 1H), 8.14 (d, J=5.3 Hz, 1H), 8.11 (d, J=8.7 Hz, 1H), 7.38 (dd, J=12.2, 2.6 Hz, 1H), 7.32 (d, J=2.6 Hz, 1H), 7.27 (d, J=1.0 Hz, 1H), 7.25 (dd, J=5.3, 1.5 Hz, 1H), 6.70 (d, J=4.9 Hz, 1H), 4.30-4.24 (m, 2H), 3.77-3.71 (m, 2H), 3.34 (s, 3H), 2.85 (d, J=4.8 Hz, 3H); ¹⁹F NMR (471 MHz, DMSO-d₆) δ ppm −123.58 (d, J=12.0 Hz); MS (ESI⁺) m/z 328 [M+H]⁺.

Preparative separations were carried out on a Waters Prep 100q SFC System, controlled by Waters MassLynx™ Software. The system consists of an open bed injector/collector, a heated column compartment including a switch for 6 columns, a CO₂-booster pump, a pump module for modifier flow. Detection was done by UV and a quadrupole mass spectrometer (Waters Aquity QDa®, ESI-ionization). To enable quantitative collection, the gas liquid separator was driven with a make-up flow of 30 mL/minute of methanol. The backpressure regulator was set to 120 bar and heated to 60° C. Columns were kept at 30° C. during the separation.

### Example 2: Preparation of [¹⁸F] Compound (I), 4-[8-(¹⁸F)fluoro-6-(2-methoxyethoxy)quinolin-2-yl]-N-methylpyridin-2-amine

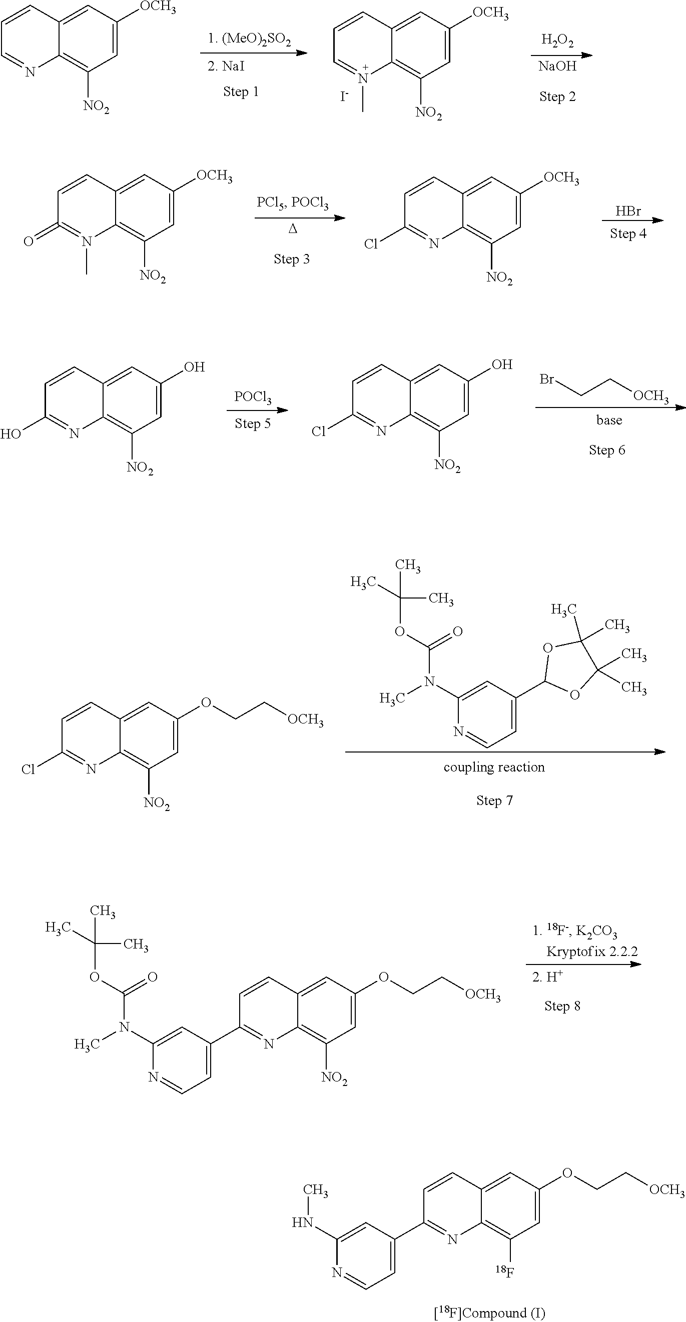

### Example 2-Step 1: 6-methoxy-1-methyl-8-nitroquinolin-1-ium iodide

6-Methoxy-8-nitroquinoline (17 g, 83 mmol) was dissolved in dimethyl sulfate (31.5 g, 250 mmol). The mixture was stirred at 100° C. for 12 hours. One additional vial was set up and run as described above, and both reaction mixtures were then combined. The combined mixture was added to water (17 mL). Then sodium iodide was added to the mixture giving a precipitate. The precipitate was collected by filtration, rinsed with water, and dried under vacuum to give the titled compound (47 g, 90% yield). ¹H NMR (400 MHz, DMSO-d₆) δ ppm 9.47 (d, J=5.5 Hz, 1H), 9.28 (d, J=8.0 Hz, 1H), 8.61 (d, J=3.0 Hz, 1H), 8.35-8.24 (m, 2H), 4.38 (s, 3H), 4.06 (s, 3H).

### Example 2-Step 2: 6-methoxy-1-methyl-8-nitroquinolin-2(1H)-one

To solution of 6-methoxy-1-methyl-8-nitroquinolin-1-ium iodide (32 g, 146 mmol) in ethanol (60 mL) was added 2 M NaOH (64 mL, 128 mmol). 30% H₂O₂ (128 mL, 1.25 mol) was slowly added at 50° C. to the mixture. When all of the peroxide had been added, the suspension was cooled and filtered. One additional vial was set up and run as described above, and both collected precipitates were then combined. The precipitates were washed with water and dried under vacuum to give the titled compound (8 g, 34.2 mmol, 23% yield). ¹H NMR (400 MHz, DMSO-d₆) δ ppm 7.98 (d, J=9.5 Hz, 1H), 7.73 (d, J=3.0 Hz, 1H), 7.67 (d, J=3.0 Hz, 1H), 6.77 (d, J=9.5 Hz, 1H), 3.87 (s, 3H), 3.27 (s, 3H).

### Example 2-Step 3: 2-chloro-6-methoxy-8-nitroquinoline

To solution of 6-methoxy-1-methyl-8-nitroquinolin-2(1H)-one (6 g, 25.6 mmol) in phosphorus oxychloride (120 mL, 1.28 μmol) was added phosphorus pentachloride (6.40 g, 30.7 mmol). The mixture was stirred at 100° C. for 3 hours. One additional vial was set up and run as described above, and both reaction mixtures were then combined. Phosphorus oxychloride was removed by distillation under reduced pressure. The crude residue was poured into water, whereupon a precipitate formed. The precipitate was collected by filtration, rinsed with water, and dried under vacuum to give the titled compound (10 g, 87% yield). ¹H NMR (400 MHz, DMSO-d₆) δ ppm 8.50 (d, J=8.6 Hz, 1H), 8.13 (d, J=2.6 Hz, 1H), 7.80 (d, J=2.6 Hz, 1H), 7.74 (d, J=8.8 Hz, 1H), 3.96 (s, 3H).

### Example 2-Step 4: 8-nitroquinoline-2,6-diol

2-Chloro-6-methoxy-8-nitroquinoline (3 g, 12.6 mmol) was dissolved in hydrobromic acid (60 mL, 442 mmol). The mixture was stirred at 130° C. for 16 hours. One additional vial was set up and run as described above, and both reaction mixtures were then combined. The combined mixture was cooled with an ice bath. Insoluble byproducts were removed from the reaction mixture by filtration and discarded. The filtrate was concentrated in vacuo to give the titled compound (6 g, 93% yield). ¹H NMR (400 MHz, DMSO-d₆) δ ppm 8.01 (br d, J=9.5 Hz, 1H), 7.84 (br s, 1H), 7.57 (br s, 1H), 6.66 (br d, J=9.0 Hz, 1H).

### Example 2-Step 5: 2-chloro-8-nitroquinolin-6-ol

8-Nitroquinoline-2,6-diol was dissolved in phosphorus oxychloride (35 mL, 375 mmol). The mixture was stirred at 100° C. for 1 hour. One additional vial was set up and run as described above, and both reaction mixtures were then combined. The reaction mixture was distilled under reduced pressure to remove phosphorus oxychloride. Then the residue was diluted with ethyl acetate (50 mL) and poured into water. Ethyl acetate (50 mL) was added, and the two phases were separated. The water phase was extracted with ethyl acetate (2×30 mL). The combined organic phases were washed with brine (30 mL), dried over anhydrous Na₂SO₄, filtered, and concentrated. The crude product was purified by column chromatography on silica gel eluted with petroleum ether/ethyl acetate=3:1. The crude residue was purified by precipitation from tert-butyl methyl ether (5 mL) and acetonitrile (0.5 mL). The mixture was filtered. The filter cake was washed with petroleum ether and dried under vacuum to give the titled compound (3.19 g, 32% yield). ¹H NMR (400 MHz, DMSO-d₆) δ ppm 10.98 (br s, 1H), 8.45 (d, J=8.8 Hz, 1H), 7.88 (d, J=2.4 Hz, 1H), 7.66 (d, J=8.8 Hz, 1H), 7.53 (d, J=2.4 Hz, 1H).

### Example 2-Step 6: 2-chloro-6-(2-methoxyethoxy)-8-nitroquinoline

To 2-chloro-8-nitroquinolin-6-ol (200 mg, 0.89 mmol) and K₂CO₃ (369 mg, 2.67 mmol) in N,N-dimethylformamide (2 mL) was slowly added 1-bromo-2-methoxyethane (0.25 mL, 2.67 mmol, 1.0 equivalent), and the mixture was stirred at room temperature overnight. The reaction mixture was poured into saturated NaHCO₃ solution and extracted with dichloromethane. Concentration of the organic fraction afforded the titled compound (280 mg, 0.89 mmol, yield 100%). MS (ESI⁺) m/z 283 [M+H]⁺.

### Example 2-Step 7: tert-butyl {4-[6-(2-methoxyethoxy)-8-nitroquinolin-2-yl]pyridin-2-yl}methylcarbamate

2-Chloro-6-(2-methoxyethoxy)-8-nitroquinoline (153 mg, 0.54 mmol), tert-butyl methyl(4-(4,4,5,5-tetramethyl-1,3,2-dioxaborolan-2-yl) pyridin-2-yl)carbamate (253 mg, 0.76 mmol, 1.4 equivalents), [1,1′-bis(diphenylphosphino)ferrocene]dichloropalladium(II), complex with dichloromethane (44 mg, 0.054 mmol, 0.1 equivalent), and Cs₂CO₃ (529 mg, 1.62 mmol, 3 equivalents) were dissolved in a microwave vial in 1,4-dioxane (4 mL) and water (0.4 mL). The reaction mixture was sparged with argon and then heated to 80° C. for 3 hours (microwave oven: Biotage® Initiator+, 400 W). The cooled crude reaction mixture was diluted with dichloromethane and insoluble material was filtered off. The organic fraction was washed with water, dried over MgSO₄, and purified by flash chromatography on silica, eluted with 0-50% ethyl acetate/cyclohexane to give the titled compound (134 mg, 0.30 mmol, yield 55%). ¹H NMR (500 MHz, DMSO-d₆) δ ppm 8.59 (d, J=8.9 Hz, 1H), 8.55 (d, J=5.2 Hz, 1H), 8.35 (d, J=8.7 Hz, 1H), 8.33 (d, J=1.6 Hz, 1H), 8.13 (d, J=2.7 Hz, 1H), 7.89 (dd, J=5.3, 1.6 Hz, 1H), 7.81 (d, J=2.7 Hz, 1H), 4.38-4.33 (m, 2H), 3.79-3.73 (m, 2H), 3.35 (s, 6H), 1.48 (s, 9H); MS (ESI⁺) m/z 455 [M+H]⁺.

### Example 2-Step 8: 4-[8-(¹⁸F)fluoro-6-(2-methoxyethoxy)quinolin-2-yl]-N-methylpyridin-2-amine, [¹⁸F] Compound (I)

[¹⁸F]Fluoride was produced in a ¹⁸O(p,n)¹⁸F reaction at Wisconsin Medical Cyclotron (Milwaukee, WI) and delivered to the radiochemistry lab on the morning of use. The [¹⁸F]fluoride was transferred to a GE TRACERlab™ FX FN synthesis module and trapped on an ion exchange cartridge (Waters Sep-Pak® Accell QMA Carbonate Plus Light cartridge, cat #WAT186004540) pre-conditioned with TRACESELECT™ Ultra ACS water (5 mL), to remove [¹⁸O]H₂O. [¹⁸F]Fluoride was eluted with 4,7,13,16,21,24-hexaoxa-1,10-diazabicyclo[8.8.8]hexacosane (Kryptofix® 2.2.2, 7.5 mg, 20 μmol) and potassium carbonate (0.75 mg, 5.4 μmol) in acetonitrile/water, Ultratrace (0.4 mL each) and transferred to the reaction vessel. The [¹⁸F]fluoride was then dried with heat (70° C.) and a stream of nitrogen or helium under full vacuum for 5 minutes followed by only full vacuum at 100° C. for 5 minutes. After drying, a solution of tert-butyl {4-[6-(2-methoxyethoxy)-8-nitroquinolin-2-yl]pyridin-2-yl}methylcarbamate (4 mg, 8.80 μmol) in anhydrous dimethyl sulfoxide (1.0 mL) was added, and the resulting solution was heated at 140° C. with stirring for 20 minutes. The reaction mixture was then cooled to 100° C. followed by the addition of 3 N HCl (1.0 mL). After stirring at 100° C. for 5 minutes to remove the tert-butoxycarbonyl protecting group, the reaction mixture was cooled to 50° C., followed by dilution with dimethyl sulfoxide (1.0 mL), 4 N NaOH (0.9 mL) and sterile water for injection (WFI, 1.0 mL). The resulting mixture was transferred into a HPLC loading vial. The content of the loop-loading vial was transferred onto a semi-preparative HPLC (Phenomenex® Gemini® NX-C18 5 μm, 110 Å 10×250 mm column using a UV setting of 275 nm) for purification using a mobile phase of 65% 10 mM ammonium acetate and 35% acetonitrile at a flow rate of 4 mL/minute.

The titled compound peak (retention time ~27 minutes) was collected into an HIPLC dilution flask and diluted with WFI (40 mL). The purified titled compound was then trapped on a pre-conditioned 50 mg Phenomenex® Strata C18-E cartridge (catalog #8B-S001-DAK) followed by washing with WFI (5 mL). The trapped titled compound was eluted with ethanol (1.0 mL) into a formulation flask followed by diluting with 0.9% USP grade sodium chloride for injection (9 mL) (Hospira, catalog #0409-4888-02). The titled compound formulation was then passed through a 13 mm 0.22 μm Millex-GV PVDF filter, (Millipore, catalog #SLGVR13SL), transferred into a sterile empty vial, and submitted for quality control testing.

Chemical and radiochemical purities/identities were analyzed using analytical HPLC (Agilent 1260) using a Phenomenex® Luna® C18(2) analytical column (5 μm, 110 Å, 4.6×150 mm, part #00F-4251-E0) eluted with a mobile phase of 55% 10 mM ammonium acetate and 45% acetonitrile at a flow rate of 1 mL/minute and a Carroll-Ramsey model 105S-1 single channel, high-sensitivity radiodetector consisting of a CsI(Tl) scintillating crystal, optically coupled to a 1 cm² silicon pin diode. The identity of the labeled compound was confirmed by co-injection of the authentic standard on HPLC. The molar activity was determined by injection of an aliquot of the final solution with known radioactivity on the analytical HPLC system described above. The area of the UV peak corresponding to the carrier product was measured and compared to a standard curve relating mass to UV absorbance. Radioactivity was measured with a Capintec CRC®-15 PET dose calibrator. Radiochemical purity for doses was >99%, and the identity was confirmed by comparing the retention time of the radiolabeled product with that of the corresponding unlabeled reference standard. The results from an average of 4 experiments are summarized below:

$$Total synthesis time : 80.8 + 1.3 minutes$$

$$Decay - corrected yield : 13.8 + 1.3 \%$$

$$Molar activity : 5980 + 1880 Ci / mmol at end of synthesis$$

$$Radiochemical purity : > 99 \%$$

### Example 3: Preparation of [³H] Compound (I), 4-[8-fluoro-6-(2-methoxyethoxy)quinolin-2-yl]-N—(³H₃)methylpyridin-2-amine

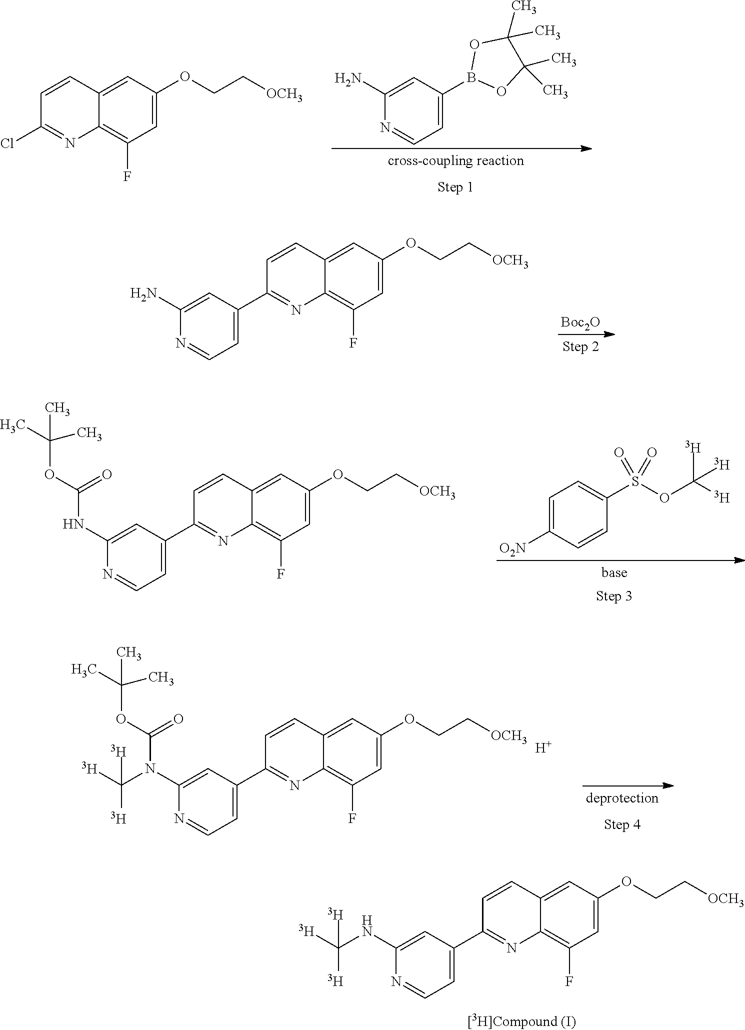

### Example 3-Step 1: 4-[8-fluoro-6-(2-methoxyethoxy)quinolin-2-yl]pyridin-2-amine

2-Chloro-8-fluoro-6-(2-methoxyethoxy)quinoline (77 mg, 0.3 mmol), 2-aminopyridine-4-boronic acid pinacol ester (79 mg, 0.36 mmol, 1.2 equivalents), [1,1′-bis(diphenylphosphino)ferrocene]dichloropalladium(II), complex with dichloromethane (24.5 mg, 0.03 mmol, 0.01 equiv.), and Cs₂CO₃ (195 mg, 0.600 mmol) were dissolved in dioxane (2 mL) and water (0.3 mL) (microwave vial). Following sparging with argon, the reaction mixture was heated to 100° C. for 2 hours (microwave oven: Biotage® Initiator+, 400 W). The crude reaction mixture was poured onto saturated NaHCO₃ solution and extracted with dichloromethane. The combined organic fractions were washed with saturated NaCl solution, dried over MgSO₄, and concentrated in vacuo. The residue was purified by flash chromatography on silica, eluted with 0-100% ethyl acetate/cyclohexane, to afford the titled compound (60 mg, 0.18 mmol, yield 60%). ¹H NMR (500 MHz, DMSO-d₆) δ ppm 8.41 (dd, J=8.8, 1.5 Hz, 1H), 8.11-8.04 (m, 2H), 7.39 (dd, J=12.2, 2.6 Hz, 1H), 7.34-7.28 (m, 2H), 7.25 (dd, J=5.4, 1.6 Hz, 1H), 6.11 (s, 2H), 4.32-4.24 (m, 2H), 3.78-3.71 (m, 2H), 3.35 (s, 3H); MS (ESI⁺) m/z 314 [M+H]⁺.

### Example 3-Step 2: tert-butyl {4-[8-fluoro-6-(2-methoxyethoxy)quinolin-2-yl]pyridin-2-yl}carbamate

4-[8-Fluoro-6-(2-methoxyethoxy)quinolin-2-yl]pyridin-2-amine (27 mg, 0.086 mmol) was dissolved in dichloromethane (5 mL) and di-tert-butyl dicarbonate (Boc₂O) (56 mg, 0.26 mmol, 3 equivalents) (dissolved in 0.5 mL dichloromethane) as well as N,N-dimethylpyridin-4-amine (31 mg, 0.26 mmol, 3 equivalents) were added at room temperature. Stirring was continued overnight to yield a mixture of starting material, tert-butoxycarbonylamino and bis-tert-butoxycarbonylamino products. Another 1.5 equivalents of di-tert-butyl dicarbonate and N,N-dimethylpyridin-4-amine were added, and stirring was continued at room temperature to achieve full conversion to the bis-tert-butoxycarbonylamino (LSMS: Column-YMC Meteoric Core C18, 50×2.1 mm, 2.7 μm; 50-100% acetonitrile/water (0.1% formic acid) over 1.8 minutes, 50° C., flow: 1 mL/minute). 1 M NaOH (3 mL) and methanol (5 mL) were added to the reaction mixture and vigorously stirred until complete hydrolysis to the desired tert-butoxycarbonylamino material was observed (LCMS). Then, the crude reaction mixture was poured onto saturated NaCl solution and extracted with dichloromethane. The combined organic fractions were dried over MgSO₄ and concentrated to dryness. The residue was pre-purified by flash chromatography on silica, eluted with 0-80% ethyl acetate/cyclohexane followed by HPLC purification (Column: Waters® XBridge™ OBD™ Prep C8, 5 μm, 150×30 mm; Eluent: (A) water+0.2% NH₄OH, (B) acetonitrile+0.2% NH₄OH; Gradient: 0-0.73 minute (50.3% A/49.7% B), 8.12 minutes (30.3% A/69.7% B), 8.13 minutes (100% B); Flow: 80 mL/minute) to afford the titled compound (20 mg, 0.046 mmol, yield 30%). ¹H NMR (500 MHz, DMSO-d₆) δ ppm 9.88 (s, 1H), 8.60 (dd, J=1.6, 0.8 Hz, 1H), 8.47 (dd, J=8.9, 1.5 Hz, 1H), 8.41 (dd, J=5.2, 0.8 Hz, 1H), 8.17 (d, J=8.7 Hz, 1H), 7.80 (dd, J=5.2, 1.6 Hz, 1H), 7.42 (dd, J=12.1, 2.6 Hz, 1H), 7.35 (d, J=2.6 Hz, 1H), 4.33-4.25 (m, 2H), 3.80-3.70 (m, 2H), 3.35 (s, 3H), 1.52 (s, 9H); ¹⁹F NMR (471 MHz, DMSO-d₆) δ ppm −123.30 (d, J=7.9 Hz); MS (ESI⁺) m/z 414 [M+H]⁺.

### Example 3-Step 3: tert-butyl {4-[8-fluoro-6-(2-methoxyethoxy)quinolin-2-yl]pyridin-2-yl}(³H₃)methylcarbamate

In a 4 mL vial containing a stir bar that was flushed with nitrogen, a suspension of tert-butyl {4-[8-fluoro-6-(2-methoxyethoxy)quinolin-2-yl]pyridin-2-yl}carbamate (1.7 mg, 4.1 μmol) in anhydrous N,N-dimethylformamide (0.2 mL) was added. To this mixture, sodium hydride (57%, 0.173 mg, 4.11 μmol) was added at 0° C., and this solution was stirred for 30 minutes at room temperature. In a separate 4 mL vial containing a stir bar, a solution of (³H₃)methyl 4-nitrobenzene-1-sulfonate (10 mCi, 0.121 μmol) in acetonitrile (0.1 mL) was added and concentrated to dryness on a rotatory evaporator. The solution of tert-butyl {4-[8-fluoro-6-(2-methoxyethoxy)quinolin-2-yl]pyridin-2-yl}carbamate was transferred to the vial containing (³H₃)methyl 4-nitrobenzene-1-sulfonate, and the mixture was stirred for 2 hours at room temperature. The reaction was worked up by first quenching with brine (0.1 mL) followed by extracting with ethyl acetate (2×3 mL). The combined organic fractions were concentrated under vacuum to afford 10 mCi (4.11 μmol) of crude titled compound.

### Example 3-Step 4: 4-[8-fluoro-6-(2-methoxyethoxy)quinolin-2-yl]-N-(³H₃)methylpyridin-2-amine, [3H] Compound (I)

Crude tert-butyl {4-[8-fluoro-6-(2-methoxyethoxy)quinolin-2-yl]pyridin-2-yl}(³H₃)methylcarbamate was dissolved in dichloromethane (0.5 mL), and this solution was transferred into a 5 mL flask containing a stir bar. Trifluoroacetic acid (0.4 mL) was added, and the solution was stirred for 20 hours at room temperature. The reaction mixture was concentrated to dryness. The crude titled compound (10 mCi) was dissolved in an acetonitrile (0.8 mL) and water (0.1 mL) mixture and used for the preparative HPLC purification.

Approximately 0.05 mL of crude titled compound solution was injected onto a Phenomenex® Luna® C18 column (5 μm, 250 mm×10 mm ID) using an Agilent 1200 series HPLC system. The titled compound was eluted at a flow rate of approximately 4.7 mL/minute with an isocratic solvent flow of 48% mobile phase B for 15 minutes, where mobile phase A=10 mM ammonium acetate and mobile phase B=acetonitrile. Peaks were detected and chromatograms were obtained using an Agilent variable wavelength UV detector set at 254 nm and ChemStation software. The fractions containing titled compound were collected around 10 minutes, using an Agilent fraction collector, pooled, and concentrated in vacuo to afford 0.3 mCi with a radiochemical purity of 95%. The molar activity of titled compound was determined to be 81.8 Ci/mmol by LCMS (LC—Agilent 1260 using Ascentis Express C18, 150×4.6 mm, 2.7 μm, flow rate of 0.8 mL/minute at 254 nm, 30° C. column temperature, with a mobile phase solution of solvents A: 0.1% formic acid in water and B: acetonitrile. A gradient 20 to 95% acetonitrile over 10 minutes and equilibrate to 20% acetonitrile for 2 minutes. Mass spec—Agilent 6130 quadrupole unit coupled with LC was used in API-ES ionization mode, authentic unlabeled Compound (I) showed a peak at m/z 327 [M+H]⁺ and [³H] Compound (I) showed a peak at m/z 333 [M+H]⁺.

### Example 4: Preparation of Compound (II), 3-(fluoromethyl)-1-{2-[2-(methylamino)pyridin-4-yl]quinolin-6-yl}azetidin-3-ol

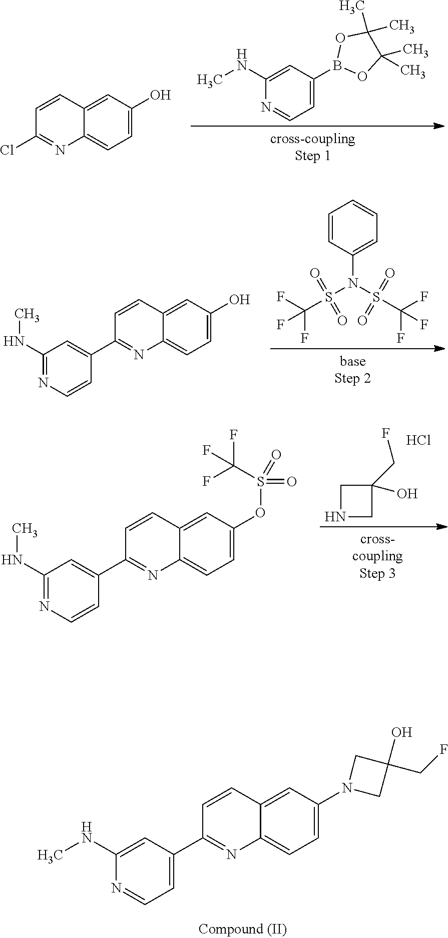

### Example 4-Step 1: 2-[2-(methylamino)pyridin-4-yl]quinolin-6-ol

2-Chloroquinolin-6-ol (453 mg, 2.52 mmol), N-methyl-4-(4,4,5,5-tetramethyl-1,3,2-dioxaborolan-2-yl)pyridine-2-amine (650 mg, 2.78 mmol, 1.1 equivalent), [1,1′-bis(diphenylphosphino)ferrocene]dichloropalladium(II), complex with dichloromethane (206 mg, 0.25 mmol, 0.1 equivalent), and Cs₂CO₃ (2.47 g, 7.57 mmol, 3 equivalents) were dissolved in 1,4-dioxane (5 mL) and water (0.8 mL) (microwave vial). The reaction mixture was sparged with argon and heated to 70° C. for 3 hours (microwave oven: Biotage® Initiator+, 400 W). The reaction mixture was diluted with ethyl acetate, and insoluble material was filtered off. The organic fraction was washed with water, dried over MgSO₄, and concentrated in vacuo to yield crude titled compound (618 mg, 2.46 mmol, yield 97%) that was used in the following step without further purification. MS (ESI⁺) m/z 252 [M+H]⁺.

### Example 4-Step 2: 2-[2-(methylamino)pyridin-4-yl]quinolin-6-yl trifluoromethanesulfonate

2-[2-(Methylamino)pyridin-4-yl]quinolin-6-ol (450 mg, 1.79 mmol) was dissolved in tetrahydrofuran (10 mL) and K₂CO₃ (742 mg, 5.37 mmol, 3 equivalents) was added. After cooling with an ice-water bath, 1,1,1-trifluoro-N-phenyl-N-(trifluoromethanesulfonyl)methanesulfonamide (640 mg, 1.79 mmol, 1 equivalent) was added, and the reaction mixture was stirred and allowed to warm to room temperature overnight. The reaction mixture was diluted with dichloromethane, poured onto saturated NaHCO₃ solution, and extracted with dichloromethane. The combined organic layers were dried over Na₂SO₄ and concentrated to dryness. The residue was purified by flash chromatography on silica, eluted with 5% methanol in dichloromethane, affording the titled compound (68 mg, 0.93 mmol, yield 52%). MS (ESI⁺) m/z 384 [M+H]⁺.

### Example 4-Step 3a: tert-butyl 3-methylideneazetidine-1-carboxylate

To a solution of methyltriphenyl phosphonium bromide (49.0 g, 137 mmol) in tetrahydrofuran (400 mL) was added potassium tert-butoxide (15.4 g, 137 mmol) at 25° C. The reaction solution was stirred at 25° C. for 1 hour, followed by the addition of tert-butyl 3-oxoazetidine-1-carboxylate (10 g, 58.4 mmol) at 25° C. The mixture was stirred at 35° C. for 2 hours. TLC (petroleum ether/ethyl acetate=4/1) showed starting material was consumed and a new spot was detected. Sixteen additional reactions were set up and run as described above. All seventeen reaction mixtures were then combined, filtered through a pad of diatomaceous earth that was washed with tetrahydrofuran (500 mL). The filtrate was washed with water (1 L), dried over sodium sulfate, filtered, and concentrated. The residue was purified by flash column chromatography eluted with petroleum ether/ethyl acetate=100/1 to 10/1 to give the titled compound (130 g, 77% yield). ¹H NMR (400 MHz, CDCl₃) δ ppm 1.40 (s, 9H), 4.43 (s, 4H), 4.94 (t, 0.1=2.43 Hz, 2H).

### Example 4-Step 3b: tert-butyl 1-oxa-5-azaspiro[2.3]hexane-5-carboxylate

To a solution of tert-butyl 3-methylideneazetidine-1-carboxylate (30 g, 177 mmol) in CHCl₃ (180 mL) was added 3-chloroperoxybenzoic acid (141 g, 515 mmol, 85% weight) at 0° C. The resulting mixture was stirred at 25° C. for 2 days. TLC (petroleum ether/ethyl acetate=4/1) showed starting material was consumed and a new spot was detected. Two additional reactions were set up and run as described above. All three reaction mixtures were then combined and quenched with a mixture of 10% sodium thiosulfate and saturated sodium bicarbonate solutions (250 mL, 1:1). The organic layer was isolated, dried over sodium sulfate, filtered, and concentrated to give the titled compound (40 g, 40.6% yield), which was used in the next step directly. ¹H NMR (400 MHz, CDCl₃) δ ppm 4.27-4.22 (m, 2H), 4.21-4.13 (m, 2H), 2.88-2.74 (m, 2H), 1.49-1.36 (m, 9H).

### Example 4-Step 3c: tert-butyl 3-(fluoromethyl)-3-hydroxyazetidine-1-carboxylate

A solution of tert-butyl 1-oxa-5-azaspiro[2.3]hexane-5-carboxylate (5 g, 27.0 mmol) in triethylamine trihydrofluoride (30 mL, 27.0 mmol) was stirred at 25° C. for 4 days. TLC (petroleum ether/ethyl acetate=2/1) showed the starting material was consumed and a new spot was detected. Seven additional reactions were set up and run as described above. All eight reaction mixtures were then combined and concentrated. The residue was diluted with ethyl acetate (1 L) and adjusted with aqueous saturated NaHCO₃ to pH=8. The mixture was extracted with ethyl acetate (3×500 mL), and the combined organic layers were washed with brine (3×500 mL), dried over Na₂SO₄, filtered, and concentrated. The residue was purified by column chromatography on silica gel (petroleum ether/ethyl acetate=50:1 to 2:1) to give the titled compound (16 g, 41.3% yield). ¹H NMR (400 MHz, CDCl₃) δ ppm 4.53 (s, 1H), 4.42 (s, 1H), 3.95-3.80 (m, 4H), 1.49-1.32 (m, 9H).

### Example 4-Step 3d: 3-(fluoromethyl)azetidin-3-ol

To a solution of tert-butyl 3-(fluoromethyl)-3-hydroxyazetidine-1-carboxylate (7 g, 34.1 mmol) in 1,4-dioxane (50 mL) was added 4 N HCl/dioxane (100 mL). The reaction solution was stirred at 25° C. for 6 hours. Another additional reaction was set up and run as described above. Both reaction mixtures were then combined and concentrated at 40° C. The residue was diluted with water (70 mL) and extracted with ethyl acetate (3×50 mL). The combined organic layers were washed with water (2×20 mL). The combined aqueous phase was lyophilized to give the titled compound (6 g, 41.4% yield) as a hydrochloride salt. ¹H NMR (400 MHz, DMSO-d₆) δ ppm 9.64-9.32 (m, 1H), 8.22 (br s, 1H), 6.76-6.29 (m, 1H), 4.65-4.38 (m, 2H), 3.88 (br t, J=5.6 Hz, 2H), 3.74 (s, 1H), 2.95 (br d, J=5.5 Hz, 1H).

### Example 4-Step 3e: 3-(fluoromethyl)-1-{2-[2-(methylamino)pyridin-4-yl]quinolin-6-yl}azetidin-3-ol, Compound (II)

2-[2-(Methylamino)pyridin-4-yl]quinolin-6-yl trifluoromethanesulfonate (300 mg, 0.78 mmol), 3-(fluoromethyl)azetidin-3-ol hydrochloride (166 mg, 1.17 mmol, 1.5 equivalents), K₃PO₄ (997 mg, 4.7 mmol, 6 equivalents), and RuPhos Pd G4 (133 mg, 0.16 mmol, 0.2 equivalent) were suspended in tetrahydrofuran (8 mL) under Ar. Following sparging with argon, the reaction mixture was capped and heated to 70-80° C. for 5 hours. The reaction mixture was diluted with dichloromethane and washed successively with water and saturated NaCl solution. The organic layer was dried over MgSO₄ and concentrated in vacuo. The residue was pre-purified by flash chromatography on silica, eluted with 0-70% ethyl acetate/cyclohexane, followed by HPLC (Column: Waters® XSelect CSH Cl 8 OBD Prep, 5 μm, 150×30 mm; Eluent: (A) water+0.1% formic acid, (B) acetonitrile+0.1% formic acid; Gradient: 0-0.1 minute (94.8% A/5.2% B), 7.40 minutes (74.8% A/25.2% B), 7.41 minutes (100% B), 9.24 minutes (100% B); Flow: 80 mL/minute) purification to afford the titled compound (68 mg, 0.177 mmol, yield 22%). ¹H NMR (500 MHz, DMSO-d₆) δ ppm 8.18 (dd, J=8.8, 0.7 Hz, 1H), 8.13-8.06 (m, 1H), 7.91 (dd, J=8.9, 6.7 Hz, 2H), 7.22 (d, J=4.9 Hz, 2H), 7.14 (dd, J=9.1, 2.6 Hz, 1H), 6.78 (d, J=2.6 Hz, 1H), 6.59 (q, J=4.8 Hz, 1H), 4.57 (d, J=47.5 Hz, 2H), 4.07 (d, J=8.2 Hz, 2H), 3.78 (dd, J=8.4, 3.0 Hz, 2H), 2.84 (d, J=4.8 Hz, 3H); ¹⁹F NMR (471 MHz, DMSO-d₆) δ ppm −226.25 (t, J=47.3 Hz); MS (ESI⁺) m/z 339 [M+H]⁺.

### Example 5: Preparation of [¹⁸F] Compound (H₁), 3-[(¹⁸F)fluoromethyl]-1-{2-[2-(methylamino)pyridin-4-yl]quinolin-6-yl}azetidin-3-ol

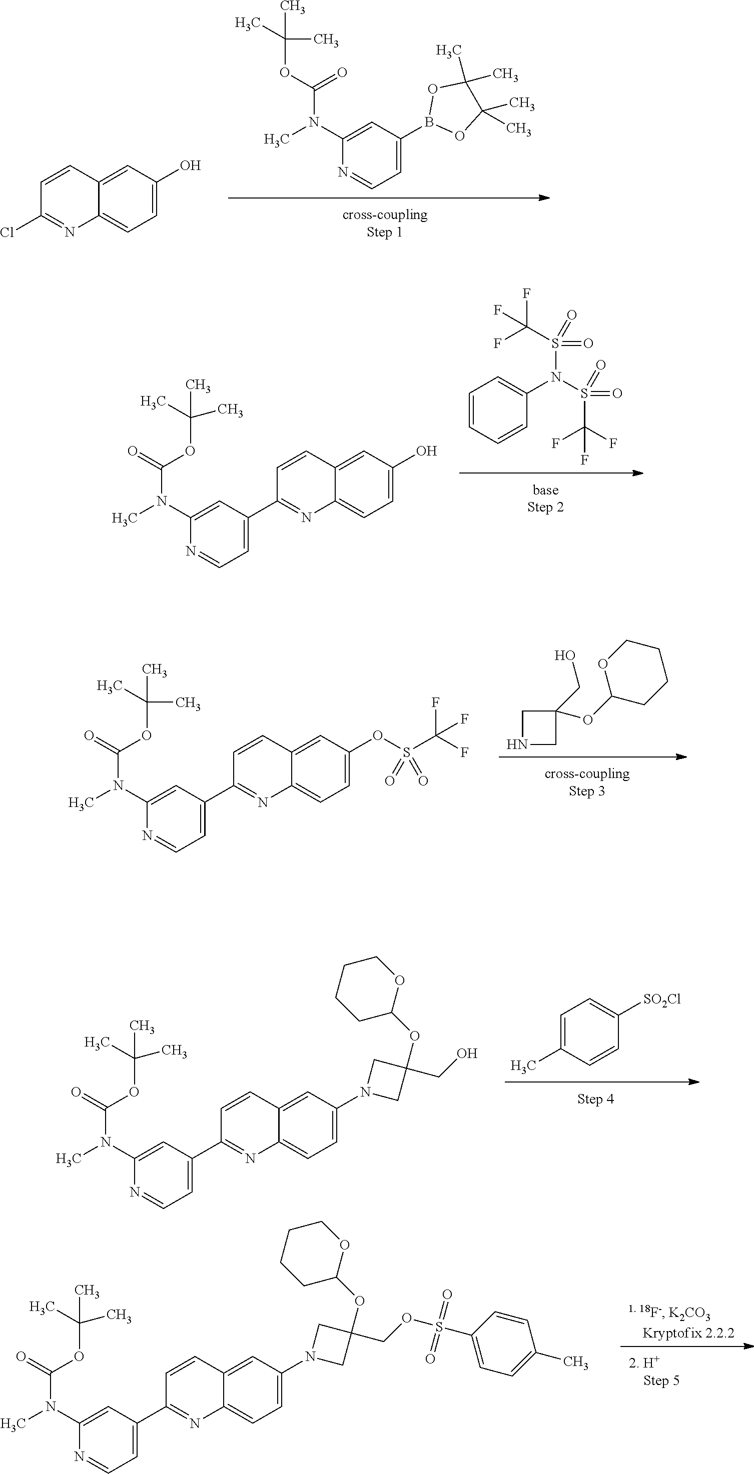

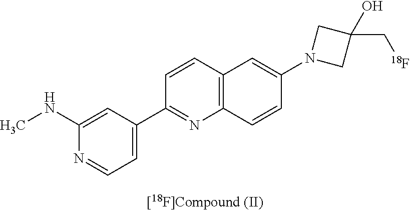 [SMILES: CNc1cc(-c2ccc3cc(N4CC(O)(C[18F])C4)ccc3n2)ccn1]

### Example 5-Step 1: tert-butyl [4-(6-hydroxyquinolin-2-yl)pyridin-2-yl]methylcarbamate

2-Chloroquinolin-6-ol (400 mg, 2.23 mmol), tert-butyl methyl(4-(4,4,5,5-tetramethyl-1,3,2-dioxaborolan-2-yl)pyridin-2-yl)carbamate (819 mg, 2.45 mmol, 1.1 equivalents), [1,1′-bis(diphenylphosphino)ferrocene]dichloropalladium(II), complex with dichloromethane (182 mg, 0.22 mmol, 0.1 equivalent), and Cs₂CO₃ (2.18 g, 6.68 mmol, 3 equivalents) were dissolved in 1,4-dioxane (8 mL) and water (0.9 mL). After sparging with argon, the reaction vial was capped, and the mixture was heated to 70° C. for 1 hour. The crude reaction mixture was diluted with dichloromethane. and insoluble material was removed by filtration. The organic layer was washed with water, dried over MgSO₄ and concentrated in vacuo. The residue was purified by flash chromatography on silica, eluted with 0-50% ethyl acetate/cyclohexane, affording the titled compound (648 mg, 1.84 mmol, yield 83%). MS (ESI⁻) m/z 352 [M+H]⁺.

### Example 5-Step 2: 2-{2-[(tert-butoxycarbonyl)(methyl)amino]pyridin-4-yl}quinolin-6-yl trifluoromethanesulfonate

tert-Butyl [4-(6-hydroxyquinolin-2-yl)pyridin-2-yl]methylcarbamate (648 mg, 1.84 mmol) and K₂CO₃ (765 mg, 5.53 mmol, 3 equivalents) were suspended in tetrahydrofuran (10 mL) and cooled with an ice-water bath. N,N-Bis(trifluoromethylsulfonyl)aniline (659 mg, 1.84 mmol, 1 equivalent) was added, and the reaction mixture was allowed to warm to room temperature. Stirring was continued for 2 hours, and the precipitate was collected by filtration and washed with dichloromethane and methanol. Drying in vacuo afforded the titled compound (671 mg, 1.39 mmol, yield 75%) that was used without further purification. MS (ESI⁺) m/z 484 [M+H]⁺.

### Example 5-Step 3a: tert-butyl 3-cyano-3-[(trimethylsilyl)oxy]azetidine-1-carboxylate

To a solution of tert-butyl 3-oxoazetidine-1-carboxylate (25 g, 146 mmol) in tetrahydrofuran (250 mL) were added trimethylsilyl cyanide (47.3 mL, 353 mmol) and lithium chloride (1.060 mg, 0.025 mmol). The reaction mixture was stirred for 12 hours at 25° C. TLC (petroleum ether/ethyl acetate=5/1) showed the starting material was consumed and a new spot was detected. An additional vial was set up, and the reaction was run as described above. The two reaction mixtures were then combined. The mixture was concentrated under reduced pressure to give the titled compound (80 g, 91% yield). ¹H NMR (400 MHz, CDCl₃) δ ppm 0.22-0.30 (m, 9H) 1.44 (s, 9H) 4.02 (d, J=9.88 Hz, 2H) 4.34 (d, J=9.88 Hz, 2H).

### Example 5-Step 3b: tert-butyl 3-cyano-3-hydroxyazetidine-1-carboxylate

To a solution of HCl/methyl alcohol (200 mL, 0.5 M) was added tert-butyl 3-cyano-3-[(trimethylsilyl)oxy]azetidine-1-carboxylate (20 g, 74.0 mmol). The reaction was stirred for 12 hours at 25° C. TLC (petroleum ether/ethyl acetate=5/1) showed the starting material was consumed, and a new spot was detected. An additional three vials were set up and run as described above. All four reaction mixtures were then combined. The mixture was concentrated under reduced pressure. The residue was purified by chromatography column on silica gel (eluted with petroleum ether/ethyl acetate=100/1 to 0/1) to give the titled compound (51 g, 78% yield). ¹H NMR (400 MHz, DMSO-d₆) δ ppm 1.39 (s, 9H) 3.89 (d, J=9.54 Hz, 2H) 4.28 (d, J=9.54 Hz, 2H) 7.52 (s, 1H); MS (ESI⁺) m/z 143.1 [M−100]⁺.

### Example 5-Step 3c: tert-butyl 3-cyano-3-[(oxan-2-yl)oxy]azetidine-1-carboxylate

To a solution of tert-butyl 3-cyano-3-hydroxyazetidine-1-carboxylate (22.5 g, 114 mmol) in dichloromethane (65 mL) were added 3,4-dihydro-2H-pyran (17.00 g, 202 mmol) and 4-methylbenzenesulfonic acid (0.344 mg, 0.002 mmol). The reaction mixture was stirred at 25° C. for 12 hours. TLC (petroleum ether/ethyl acetate=5/1) showed the starting material was consumed, and a new spot was detected. An additional one vial was set up and run as described above. The two reaction mixtures were then combined and concentrated under reduced pressure. The residue was purified by column chromatography on silica gel (eluted with petroleum ether/ethyl acetate=100/1 to 5/1) to give the titled compound (45 g, 63.2% yield). ¹H NMR (400 MHz, CDCl₃) δ ppm 1.45 (s, 9H) 1.55-1.67 (m, 4H) 1.73-1.90 (m, 2H) 3.52-3.65 (m, 1H) 3.94 (ddd, J=10.82, 7.00, 3.39 Hz, 1H) 4.15 (d, J=9.54 Hz, 1H) 4.21-4.32 (m, 2H) 4.35 (dd, J=9.54, 0.63 Hz, 1H) 4.96-5.05 (m, 1H); MS (ESI⁺) m/z 227.1 [M−100]⁺.

### Example 5-Step 3d: 1-(tert-butoxycarbonyl)-3-[(oxan-2-yl)oxy]azetidine-3-carboxylic acid

To a solution of tert-butyl 3-cyano-3-[(oxan-2-yl)oxy]azetidine-1-carboxylate (22.5 g, 80 mmol) in ethanol (200 mL) and water (200 mL) was added KOH (17.88 g, 319 mmol). The reaction mixture was stirred at 100° C. for 12 hours. TLC (petroleum ether/ethyl acetate=5/1) showed the starting material was consumed and a new spot was detected. An additional one vial was set up and run as described above. After cooling to 25° C., the two reaction mixtures were then combined. The ethanol was removed under reduced pressure, and the water phase was acidified with 2 N HCl to pH=5 and extracted with ethyl acetate (3×200 mL). The combined organic layers were dried over Na₂SO₄, filtered, and concentrated under reduced pressure to give the titled compound (35 g, 65.6% yield). ¹H NMR (400 MHz, DMSO-d₆) δ ppm 1.38 (s, 9H) 1.42-1.55 (m, 4H) 1.64-1.78 (m, 2H) 3.37-3.46 (m, 1H) 3.79-3.94 (m, 2H) 3.99-4.20 (m, 3H) 4.74 (br d, J=1.38 Hz, 1H) 12.84 (br s, 1H); MS (ESI⁺) m/z 202.1 [M−100]⁺.

### Example 5-Step 3e: tert-butyl 3-(hydroxymethyl)-3-[(oxan-2-yl)oxy]azetidine-1-carboxylate

To a solution of 1-(tert-butoxycarbonyl)-3-[(oxan-2-yl)oxy]azetidine-3-carboxylic acid (7.5 g, 24.89 mmol) in tetrahydrofuran (60 mL) was added N,N-carbonyldiimidazole (4.44 g, 27.4 mmol), and the mixture was stirred for 10 minutes at 25° C. Then a solution of NaBH₄ (1.883 g, 49.8 mmol) in water (30 mL) was added. The reaction mixture was stirred at 25° C. for 12 hours. TLC (petroleum ether/ethyl acetate=1/1) showed the starting material was consumed and a new spot was detected. An additional one vial was set up and run as described above. The two reaction mixtures were then combined, ethyl acetate (300 mL) was added, and the organic layer was separated. The organic fraction was washed with 1 N NaHCO₃ solution (200 mL) and brine (200 mL), dried over Na₂SO₄, and concentrated under reduced pressure. The reside was purified by column chromatography on silica gel (eluted with petroleum ether/ethyl acetate=100/1 to 1/1) to give the titled compound (5 g, 66.4% yield) as colorless oil. ¹H NMR (400 MHz, CDCl₃) h ppm 1.45 (s, 9H) 1.51-1.67 (m, 4H) 1.72-1.94 (m, 2H) 3.43-3.60 (m, 2H) 3.70-3.89 (m, 6H) 3.92-4.06 (m, 1H) 4.55-4.69 (m, 1H); MS (ESI⁺) m/z 188.1 [M+1]⁺.

### Example 5-Step 3f: {3-[(oxan-2-yl)oxy]azetidin-3-yl}methanol

tert-Butyl 3-(hydroxymethyl)-3-[(oxan-2-yl)oxy]azetidine-1-carboxylate (177 mg, 0.62 mmol) and KOH (104 mg, 1.85 mmol, 3 equivalents) were dissolved in water (2 mL) and methanol (3 mL) in a microwave vial. The reaction mixture was heated to 100-120° C. (microwave oven: Biotage® Initiator+, 400 W) for six hours with additional equivalents of KOH were added in portions (up to 10 equivalents in total) until complete tert-butoxycarbonyl deprotection was observed by LCMS (Column-YMC Meteoric Core C18, 50×2.1 mm, 2.7 μm; 50-100% acetonitrile/water (0.1% formic acid) over 1.8 minutes, 50° C., flow: 1 mL/minute). The crude mixture was passed manually through a Chromabond® C18 cartridge, running water first to reduce overall salt loading before switching to water/methanol (1:1) to collect product-containing fractions. The combined water/methanol fractions were concentrated under reduced pressure at 50° C., and the residue was transferred with methanol. Concentration in vacuo afforded the titled compound (132 mg, 0.612 mmol, crude yield 100%) that was used without further purification. MS (ESI⁺) m/z 188 [M+H]⁻.

### Example 5-Step 3g: tert-butyl [4-(6-{3-(hydroxymethyl)-3-[(oxan-2-yl)oxy]azetidin-1-yl}quinolin-2-yl)pyridin-2-yl]methylcarbamate

A microwave vial was charged with 2-{2-[(tert-butoxycarbonyl)(methyl)amino]pyridin-4-yl}quinolin-6-yl trifluoromethanesulfonate (163 mg, 0.34 mmol), K₃PO₄ (340 mg, 1,602 mmol) and tBuXPhos Pd G3 (26 mg, 0.032 mmol, 0.1 equivalent). {3-[(Oxan-2-yl)oxy]azetidin-3-yl}methanol (60 mg, 0.32 mmol, 1 equivalent) dissolved in 1,4-dioxane (2 mL) and N,N-dimethylformamide (0.5 mL) was added. Following sparging with argon, the reaction vial was capped, and the reaction mixture was heated to 100° C. for 4 hours. The crude reaction mixture was poured onto water and extracted with dichloromethane. The combined organic extracts were dried over MgSO₄ and concentrated in vacuo. The residue was purified by flash chromatography on silica, eluted with 0-60% ethyl acetate/cyclohexane, to afford the titled compound (72 mg, 0.138 mmol, yield 43%). MS (ESI⁺) m/z 521 [M+H]⁻.

### Example 5-Step 4: {1-(2-{2-[(tert-butoxycarbonyl)(methyl)amino]pyridin-4-yl}quinolin-6-yl)-3-[(oxan-2-yl)oxy]azetidin-3-yl}methyl 4-methylbenzene-1-sulfonate

tert-Butyl [4-(6-{3-(hydroxymethyl)-3-[(oxan-2-yl)oxy]azetidin-1-yl}quinolin-2-yl)pyridin-2-yl]methylcarbamate (72 mg, 0.138 mmol) was dissolved in dichloromethane (3 mL). N,N-Dimethylpyridin-4-amine (25 mg, 0.21 mmol, 1.5 equivalents) and p-toluenesulfonyl chloride (30 mg, 0.16 mmol, 1.15 equivalents) were added, and the resultant reaction mixture was stirred at room temperature overnight. Another 0.5 equivalent of both reagents were added and stirring was continued for 3 days to reach complete conversion. The crude reaction mixture was poured onto 10% citric acid solution and extracted with dichloromethane. The combined organic extracts were dried over MgSO₄ and concentrated in vacuo. The residue was purified by flash chromatography on silica, eluted with 0-20% ethyl acetate/dichloromethane to afford the titled compound (45 mg, 0.067 mmol, yield 48%). ¹H NMR (500 MHz, CDCl₃) δ ppm 8.49 (d, J=5.2 Hz, 1H), 8.39-8.35 (m, 1H), 8.01 (t, J=7.8 Hz, 2H), 7.85-7.74 (m, 4H), 7.34 (d, J=8.1 Hz, 2H), 6.99 (dd, J=9.1, 2.6 Hz, 1H), 6.59 (d, J=2.6 Hz, 1H), 4.81 (dd, J=4.8, 2.8 Hz, 1H), 4.47 (d, J=10.4 Hz, 1H), 4.39 (d, J=10.4 Hz, 1H), 4.05-3.94 (m, 4H), 3.89 (ddd, J=11.0, 6.9, 3.4 Hz, 1H), 3.46 (s, 3H), 3.45-3.40 (m, 1H), 2.44 (s, 3H), 1.85-1.75 (m, 1H), 1.73-1.64 (m, 1H), 1.56 (s, 9H), 1.61-1.46 (m, 4H); MS (ESI⁺) m/z 675 [M+H]⁺.

### Example 5-Step 5: 3-[(¹⁸F)fluoromethyl]-1-{2-[2-(methylamino)pyridin-4-yl]quinolin-6-yl}azetidin-3-ol, [¹⁸F] Compound (II)

[¹⁸F]Fluoride was produced in a ¹⁸O(p,n)¹⁸F reaction at Wisconsin Medical Cyclotron (Milwaukee, WI) and delivered to the radiochemistry lab on the morning of use. The [¹⁸F]fluoride in ~1 to 4 mL of [¹⁸O]H₂O was transferred to a GE TRACERlab™ FX FN synthesis module and trapped on an ion exchange cartridge (Waters Sep-Pak® Accell QMA Carbonate Plus Light cartridge, cat #WAT186004540) pre-conditioned with TRACESELECT™ Ultra ACS water (5 mL), to remove [¹⁸O]H₂O. [¹⁸F]Fluoride was eluted into the reaction vessel by passing K₂CO₃ (0.75 mg, 5.43 μmol) in Ultratrace-grade water (0.4 mL) and 4,7,13,16,21,24-hexaoxa-1,10-diazabicyclo[8.8.8]hexacosane (Kryptofix® 2.2.2, 7.5 mg, 19.9 μmol) in acetonitrile (0.4 mL) through the cartridge. The [¹⁸F]fluoride was then dried by heat (70° C.) and a stream of nitrogen or helium under full vacuum for 5 minutes followed by only full vacuum at 100° C. for 5 minutes. After drying, a solution of {1-(2-{2-[(tert-butoxycarbonyl)(methyl)amino]pyridin-4-yl}quinolin-6-yl)-3-[(oxan-2-yl)oxy]azetidin-3-yl}methyl 4-methylbenzene-1-sulfonate (2 mg, 2.96 μmol) in anhydrous dimethyl sulfoxide (1.0 mL) was added, and the resulting solution was heated at 100° C. with stirring for 10 minutes. To the reaction mixture was added 1 N HCl (1.0 mL) followed by heating at 100° C. for 3 minutes to remove the tert-butoxycarbonyl and tetrahydropyranyl protecting groups. The reaction mixture was then cooled to 50° C. followed by neutralization with 1 N NaOH (1.1 mL) and sterile water for injection (WFI, 2.0 mL). The resulting mixture was transferred into an HPLC loading vial. The content of the loop-loading vial was transferred onto the semi-preparative HPLC (Phenomenex® Gemini® NX-C18 5 μm, 110 Å 10×250 mm column using a UV setting of 260 nm) for purification using a mobile phase of 72% 10 mM ammonium acetate and 28% acetonitrile at a flow rate of 4 mL/minute.

The titled compound peak (retention time ~26 minutes) was collected into an HPLC dilution flask and diluted with WFI (40 mL). The purified titled compound was then trapped on a pre-conditioned 50 mg Phenomenex® Strata C18-E cartridge (catalog #8B-S001-DAK) followed by washing with WFI (5 mL). The trapped titled compound was eluted with ethanol (1.0 mL) into a formulation flask followed by diluting with 0.9% USP grade sodium chloride for injection (9 mL) (Hospira, catalog #0409-4888-02). The titled compound formulation was then passed through a 13 mm 0.22 μm Millex-GV PVDF filter, (Millipore, catalog #SLGVR13SL), transferred into a sterile empty vial, and submitted for quality control testing.

Chemical and radiochemical purities/identities were analyzed using analytical HPLC (Agilent 1260) using a Phenomenex® Luna® C18(2) analytical column (5 μm, 110 Å, 4.6×150 mm, part #00F-4251-E0) eluted with a mobile phase of 65% 10 mM ammonium acetate and 35% acetonitrile at a flow rate of 1 mL/minute and a Carroll-Ramsey model 105S-1 single channel, high-sensitivity radiodetector consisting of a CsI(Tl) scintillating crystal, optically coupled to a 1 cm² silicon pin diode. The identity of the labeled compound was confirmed by co-injection of the authentic standard on HPLC. The molar activity was determined by injection of an aliquot of the final solution with known radioactivity on the analytical HPLC system described above. The area of the UV peak corresponding to the carrier product was measured and compared to a standard curve relating mass to UV absorbance. Radioactivity was measured with a Capintec CRC®-15 PET dose calibrator. Radiochemical purity for doses was >99%, and the identity was confirmed by comparing the retention time of the radiolabeled product with that of the corresponding unlabeled reference standard. The results from an average of 4 experiments are summarized below:

$$Total synthesis time : 66.8 + 0.4 min$$

$$Decay - corrected yield : 35.3 + 3. \%$$

$$Molar activity : 2649 + 944 Ci / mmol @ E O S$$

$$Radiochemical purity : > 99 \%$$

### Example 6: Preparation of [³H] Compound (II), 3-(fluoromethyl)-1-(2-{2-[(³H₃)methylamino]pyridin-4-yl}quinolin-6-yl)azetidin-3-ol

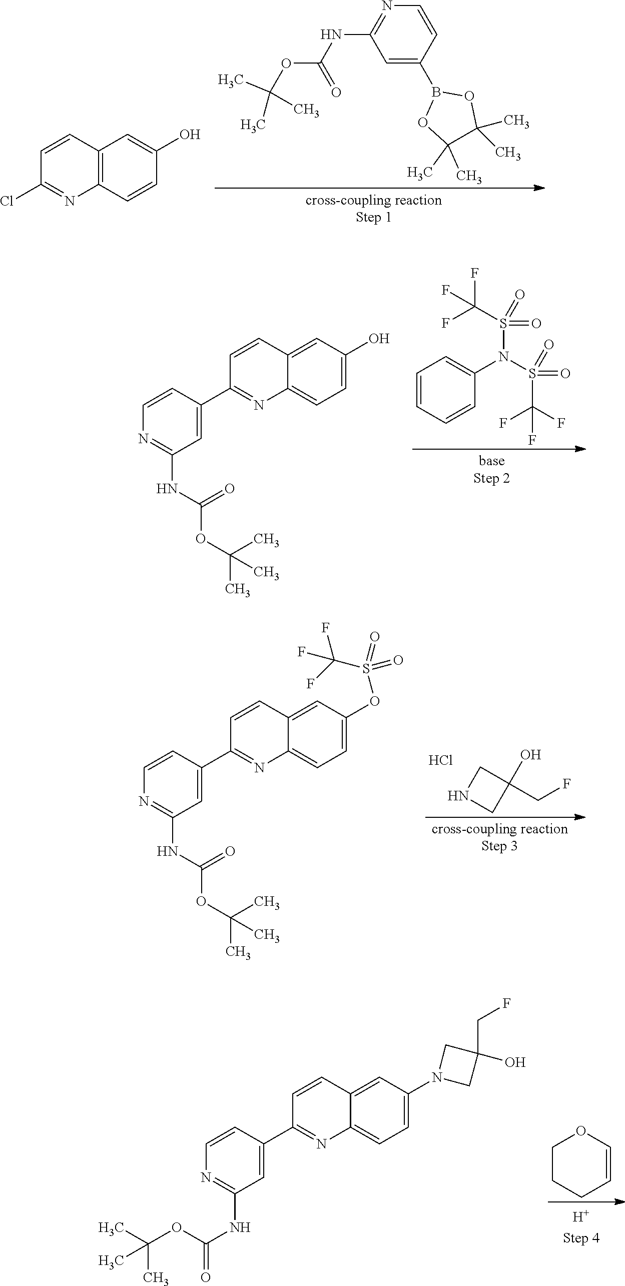

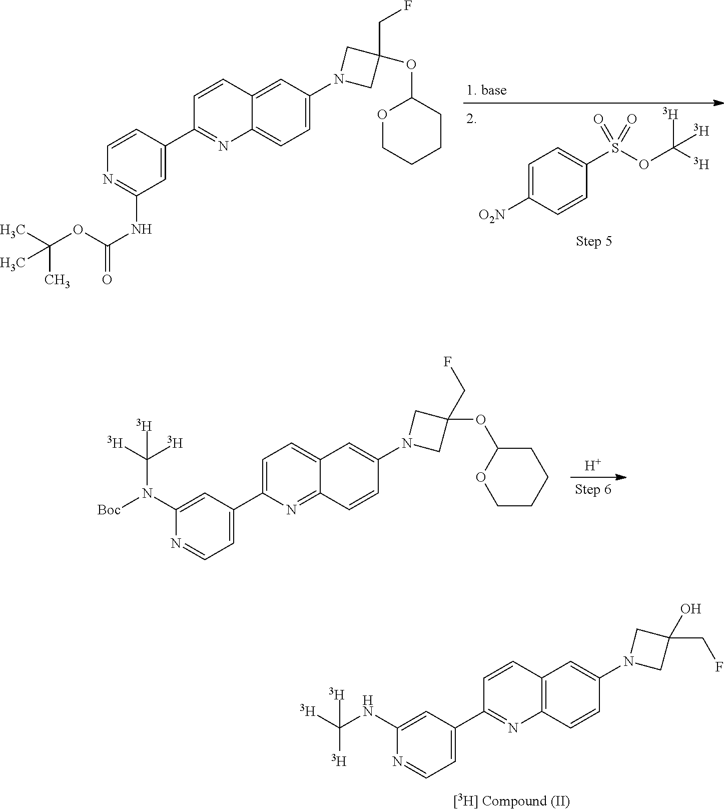

### Example 6-Step 1: tert-butyl [4-(6-hydroxyquinolin-2-yl)pyridin-2-yl]carbamate

2-Chloroquinolin-6-ol (200 mg, 1.11 mmol), tert-butyl (4-(4,4,5,5-tetramethyl-1,3,2-dioxaborolan-2-yl)pyridin-2-yl)carbamate (465 mg, 1.45 mmol, 1.3 equivalents), [1,1′-bis(diphenylphosphino)ferrocene]dichloropalladium(II), complex with dichloromethane (91 mg, 0.11 mmol, 0.1 equivalent) and Cs₂CO₃ (1.1 g, 3.34 mmol, 3 equivalents) were dissolved in 1,4-dioxane (8 mL) and water (0.9 mL) in a microwave vial. Following sparging with argon, the vial was capped, and the reaction mixture was heated to 80° C. for 3 hours. The crude reaction mixture was diluted with dichloromethane and insoluble material was removed by filtration. The organic filtrate was washed with water, dried over MgSO₄, and concentrated in vacuo. The residue was purified by flash chromatography on silica, eluted with 0-40% ethyl acetate/cyclohexane, affording the titled compound (251 mg, 0.74 mmol, yield 67%). MS (ESI⁺) m/z 338 [M+H]⁺.

### Example 6-Step 2: 2-{2-[(tert-butoxycarbonyl)amino]pyridin-4-yl}quinolin-6-yl trifluoromethanesulfonate

tert-Butyl [4-(6-hydroxyquinolin-2-yl)pyridin-2-yl]carbamate (251 mg, 0.74 mmol) and K₂CO₃ (308 mg, 2.23 mmol, 3 equivalents) were suspended in tetrahydrofuran and cooled with an ice-water bath. N,N-Bis(trifluoromethylsulfonyl)aniline (266 mg, 0.74 mmol, 1 equivalent) was added, and the reaction mixture was allowed to warm to room temperature and stirred overnight. The precipitate was filtered off and washed with dichloromethane and methanol. Drying in vacuo afforded the titled compound (380 mg, 0.73 mmol, yield 98%) that was used without further purification. MS (ESI⁺) m/z 470 [M+H]⁺.

### Example 6-Step 3: tert-butyl (4-{6-[3-(fluoromethyl)-3-hydroxyazetidin-1-yl]quinolin-2-yl}pyridin-2-yl)carbamate

2-{2-[(tert-Butoxycarbonyl)amino]pyridin-4-yl}quinolin-6-yl trifluoromethanesulfonate (211 mg, 0.45 mmol), 3-(fluoromethyl)azetidin-3-ol hydrochloride (127 mg, 0.90 mmol, 2 equivalents), K₃PO₄ (572 mg, 2.70 mmol, 6 equivalents), tBuXPhos Pd G3 (71 mg, 0.09 mmol, 0.2 equivalent) and RuPhos Pd G4 (76 mg, 0.09 mmol, 0.2 equivalent) were suspended in tetrahydrofuran (5 mL) in a microwave vial. Following sparging with argon, the vial was capped, and the reaction mixture was heated to 80° C. for 16 hours (microwave oven, Biotage® Initiator+, 400 W). The crude reaction mixture was poured onto saturated NaHCO₃ and extracted with ethyl acetate. The combined extracts were dried over MgSO₄ and concentrated under reduced pressure. The residue was purified by flash chromatography on silica, eluted with 0-10% ethyl acetate-methanol (4:1)/dichloromethane, to afford the titled compound (58 mg, 0.137 mmol, yield 30%). MS (ESI⁺) m/z 425 [M+H]⁺.

### Example 6-Step 4: tert-butyl [4-(6-{3-(fluoromethyl)-3-[(oxan-2-yl)oxy]azetidin-1-yl}quinolin-2-yl)pyridin-2-yl]carbamate

tert-Butyl (4-{6-[3-(fluoromethyl)-3-hydroxyazetidin-1-yl]quinolin-2-yl}pyridin-2-yl)carbamate (58 mg, 0.137 mmol) was dissolved in tetrahydrofuran (2 mL). 3,4-Dihydro-2H-pyran (0.135 mL, 1.37 mmol, 10 equivalents) and pyridinium p-toluenesulfonate (7 mg, 0.027 mmol, 0.2 equivalent) were then added. The reaction mixture was heated to 50-60° C. until complete conversion (LCMS. Column-YMC Meteoric Core C18, 50×2.1 mm, 2.7 μm; 50-100% acetonitrile/water (0.1% formic acid) over 1.8 minutes, 50° C., flow: 1 mL/minute). Addition of further equivalents of 3,4-dihydro-2H-pyran might be required. The crude reaction mixture was concentrated in vacuo, and the residue was purified by flash chromatography on silica, eluted with 0-10% ethyl acetate/dichloromethane, to afford the titled compound (48 mg, 0.094 mmol, yield 69%). ¹H NMR (500 MHz, DMSO-d₆) δ ppm 9.84 (s, 1H), 8.59 (dd, J=1.6, 0.8 Hz, 1H), 8.36 (dd, J=5.2, 0.8 Hz, 1H), 8.26-8.20 (m, 1H), 7.99 (d, J=8.7 Hz, 1H), 7.95 (d, J=9.1 Hz, 1H), 7.76 (dd, J=5.2, 1.6 Hz, 1H), 7.18 (dd, J=9.1, 2.6 Hz, 1H), 6.83 (d, J=2.6 Hz, 1H), 4.98 (dd, J=5.1, 2.7 Hz, 1H), 4.88-4.67 (m, 2H), 4.07 (t, J=8.7 Hz, 2H), 4.03-3.98 (m, 2H), 3.87 (ddd, J=11.1, 7.2, 3.8 Hz, 1H), 3.49 (ddd, J=10.9, 6.4, 4.0 Hz, 1H), 2.53-2.51 (m, 1H), 1.82-1.64 (m, 2H), 1.52 (s, 9H), 1.56-1.41 (m, 3H); ¹⁹F NMR (471 MHz, DMSO-d₆) δ ppm −226.78 (t, J=47.0 Hz); MS (ESI⁺) m/z 509 [M+H]⁺.

### Example 6-Step 5: tert-butyl [4-(6-{3-(fluoromethyl)-3-[(oxan-2-yl)oxy]azetidin-1-yl}quinolin-2-yl)pyridin-2-yl](³H₃)methylcarbamate

To a 4 mL septum capped vial were added sodium hydride (0.5 mg, 13 μmol) and tert-butyl [4-(6-{3-(fluoromethyl)-3-[(oxan-2-yl)oxy]azetidin-1-yl}quinolin-2-yl)pyridin-2-yl]carbamate (1.0 mg, 2 μmol). The vial was evacuated under vacuum and backfilled with N₂ three times. Anhydrous N,N-dimethylformamide (0.15 mL) was then added, and the reaction was stirred at room temperature for 30 minutes. In a separate 4 mL septum capped vial, (³H₃)methyl 4-nitrobenzene-1-sulfonate solution (10 mCi, 77 Ci/mmol) in acetonitrile was obtained, and the solvent was removed in vacuo. The vial containing dry (³H₃)methyl 4-nitrobenzene-1-sulfonate was then transferred to a Schlenk manifold and was evacuated under vacuum and backfilled with nitrogen three times. The N,N-dimethylformamide solution containing tert-butyl [4-(6-{3-(fluoromethyl)-3-[(oxan-2-yl)oxy]azetidin-1-yl}quinolin-2-yl)pyridin-2-yl]carbamate was then transferred to this vial, and the reaction was stirred at room temperature for 16 hours. (³H₃)Methyl 4-nitrobenzene-1-sulfonate was fully consumed as determined by HPLC analysis (column—Phenomenex® Luna® C18, 25 mm×4.6 mm, 5 μm, flow rate 1.5 mL/minute at 254 nm, column temp 40° C., gradient 10 to 50% acetonitrile over 9 minutes, ramp to 95% acetonitrile in 1 minute, hold at 95% acetonitrile for 3 minutes and equilibrate to 10% acetonitrile for 3 minutes as mobile phase B, whereas mobile phase A is 0.1% trifluoroacetic acid in water). The reaction mixture was then diluted with dichloromethane (4 mL), quenched with brine (0.25 mL), and then the phases were separated. The aqueous phase was extracted with additional dichloromethane (4 mL). The organic fractions were combined, and the solution was dried by rotary evaporation to give the titled compound.

### Example 6-Step 6: 3-(fluoromethyl)-1-(2-{2-[(³H₃)methylamino]pyridin-4-yl}quinolin-6-yl)azetidin-3-ol, [3H] Compound (II)

The crude tert-butyl [4-(6-{3-(fluoromethyl)-3-[(oxan-2-yl)oxy]azetidin-1-yl}quinolin-2-yl)pyridin-2-yl](³H₃)methylcarbamate was dissolved in dichloromethane (0.5 mL), trifluoroacetic acid (0.3 mL) was added, and the mixture was stirred at room temperature for 4 hours. HPLC analysis showed completion of the reaction (HPLC condition: Phenomenex® Luna® C18, 25 mm×4.6 mm, 5 μm, flow rate 1.5 mL/minute at 254 nm, column temp 40° C., gradient 10 to 50% acetonitrile over 9 minutes, ramp to 95% acetonitrile in 1 minute, hold at 95% acetonitrile for 3 minutes and equilibrate to 10% acetonitrile for 3 minutes as mobile phase B, whereas mobile phase A is 0.1% trifluoroacetic acid in water). The reaction mixture was concentrated by rotary evaporation, and the crude residue was dissolved in 1.5 mL of a 2:1 water (10 mM ammonium acetate)/acetonitrile solution for HPLC purification. The crude solution had an activity of 6.1 mCi.

The crude solution was purified by semi-preparative high performance liquid chromatography (Agilent 1260 using a Phenomenex® Luna® C18 column (250×10 mm, 5 m), 40° C. column temperature with a mobile phase solution of solvents A: 10 mM ammonium acetate and B: acetonitrile at a flow rate of 4.5 mL/minute. The crude material was purified in two runs using a mobile phase gradient of 30-50% acetonitrile over 16 minutes. The 3-(fluoromethyl)-1-(2-{2-[(³H₃)methylamino]pyridin-4-yl}quinolin-6-yl)azetidin-3-ol began eluting at 9.3 minutes and was isolated with an Agilent fraction collector. The purified fractions were combined, and the acetonitrile was removed by rotary evaporation, resulting in a 6.2 mL solution of purified 3-(fluoromethyl)-1-(2-{2-[(³H₃)methylamino]pyridin-4-yl}quinolin-6-yl)azetidin-3-ol in 10 mM ammonium acetate. To this solution was added a 2 mL ethanol solution which had 16 mg ascorbic acid as a stabilizer. The total activity was 3.1 mCi with a radiochemical purity of 95%. The molar activity as determined by LCMS (Ascentis® Express C18, 2.7 μm, 4.6×150 mm with flow of 1.4 mL/minute at 254 nm, column temperature 30° C., gradient 10 to 50% acetonitrile over 8 minutes, ramp to 95% and hold at 95% acetonitrile for 2.5 minutes and equilibrate to 10% acetonitrile for 2.5 minutes as mobile phase B, whereas mobile phase A is 0.1% formic acid in water) was 76.1 Ci/mmol.

### Example 7. In Vitro Biology Studies

In the in vitro biology studies, the following abbreviations were commonly used:

| | |
|---|---|
| AD | Alzheimer's Disease |
| BSA | Bovine serum albumin |
| DPBS | Dulbecco's phosphate buffered saline |
| FF | Flash frozen tissue blocks |
| FFPE | Formalin-fixed, paraffin-embedded tissue |
| | blocks |
| MCD | Micro-densitometry |
| NSB | Non-specific binding of radioligand |
| PSP | Progressive supranuclear palsy |
| TB | Total binding of radioligand |

Brain Tissue. Flash-frozen (FF) tissue blocks, and, for some cases, the corresponding mirrored, formalin-fixed, paraffin-embedded (FFPE) tissue blocks from the globus pallidus and putamen of normal or Progressive Supranuclear Palsy (PSP) brains were purchased from Tissue Solutions Ltd (TS; Glasgow, UK). Both FF and FFPE tissue blocks were used to assess the distribution pattern of [3H] Compound (I) and [3H] Compound (II) binding to normal and PSP brain samples by means of in vitro autoradiography. Only FF tissue blocks were used to determine the affinity of [³H] Compound (I) and [³H] Compound (II) for PSP brain by means of in vitro autoradiography. A single FF tissue block from the frontal cerebral cortex of an Alzheimer's Disease (AD) Braak stage V brain, purchased from Analytical Biological Services Inc. (ABS; Wilmington, DE, USA), was used to measure compounds binding affinity to AD-tau and amyloid-beta (A$) protein aggregates by means of radioligand binding on tissue homogenates.

Brain tissue preparation. For in vitro autoradiography experiments, FF tissue blocks were cut in 14 μm thick brain sections using a CM3050 S Cryostat (object temperature: −18° C.; chamber temperature: −20° C.) from Leica (Wetzlar, DE) mounted on Superfrost™ Plus glass slides (Gerhard Menzel GmbH Braunschweig, DE) and stored at −80° C. until the day of the experiment. FFPE blocks were cut in 14 μm thick brain sections at ambient temperature, using an HM 355S Microtome (object temperature: 4° C.) from ThermoFisher Scientific (Waltham, MA, USA), mounted on glass slides and stored at ambient temperature until the day of the experiment. On the day of the experiment, the FF brain sections were brought to ambient temperature, at least 1 hour before the incubation with the radioligand. On the day of the experiment, the FFPE slices underwent the following de-paraffination and rehydration procedure before incubation with the radioligand: 30 minutes at 70° C.; 2×10 minutes in xylene at ambient temperature; 2×10 minutes in 100% ethanol at ambient temperature; 2 minutes in 96% ethanol at ambient temperature, one minute in 70% ethanol at ambient temperature, one minute in Milli-Q® water at ambient temperature, 5 minutes in Dulbecco's phosphate buffered saline (DPBS) at ambient temperature.

The AD brain tissue block selected for the radioligand binding studies was brought up to ambient temperature, homogenized in ice cold DPBS buffer without calcium and magnesium, pH 7.4, divided into aliquots and stored at −80° C. until the day of the experiment.

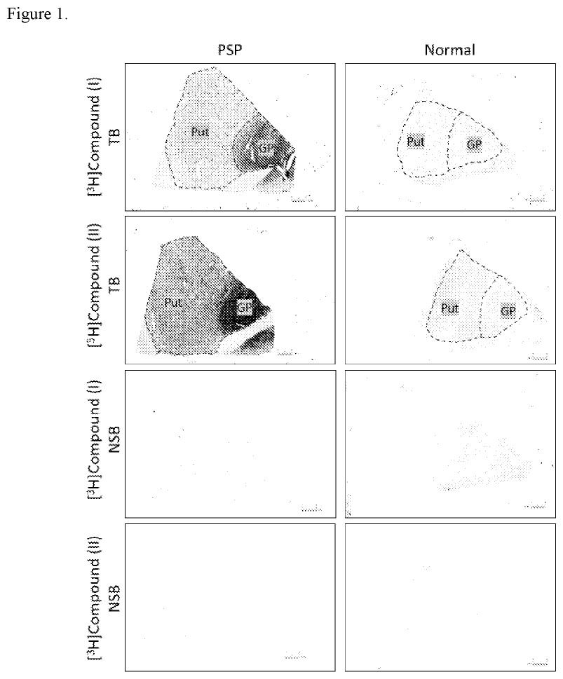

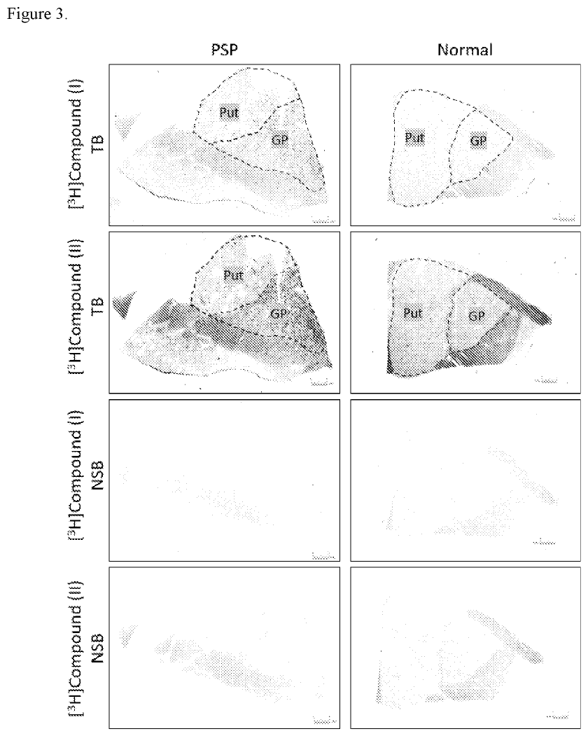

In vitro autoradiography. For the in vitro autoradiography of [3H] Compound (I) and [3H] Compound (II), 14 μm-thick FF brain slices or 14 μm-thick, de-paraffinized FFPE brain slices were incubated (90 minutes, ambient temperature) with 3 nM radioligand in DPBS buffer containing 0.1% bovine serum albumin (BSA), in the presence or absence of the corresponding non-radiolabeled compounds. The radioligand binding was stopped by washing (3×3 minutes) the tissue slices in ice-cold DPBS buffer. After rinsing (2×5 seconds) in ice-cold Milli-Q® water, the slices were air dried and placed against Tritium-sensitive BAS-TR2025 imaging plates (Fuji Film Corporation, Tokyo, JP) for seven days. The plates were read using a Phosphoimager Typhoon™ FLA-70001P (GE Healthcare, Chicago, IL USA). Total binding (TB) was defined as radioligand binding in the presence of the radioligand alone; non-specific binding (NSB) was defined as radioligand binding in the presence of 10 μM of the corresponding non-radiolabeled compound (FIG. 1, FIG. 3).

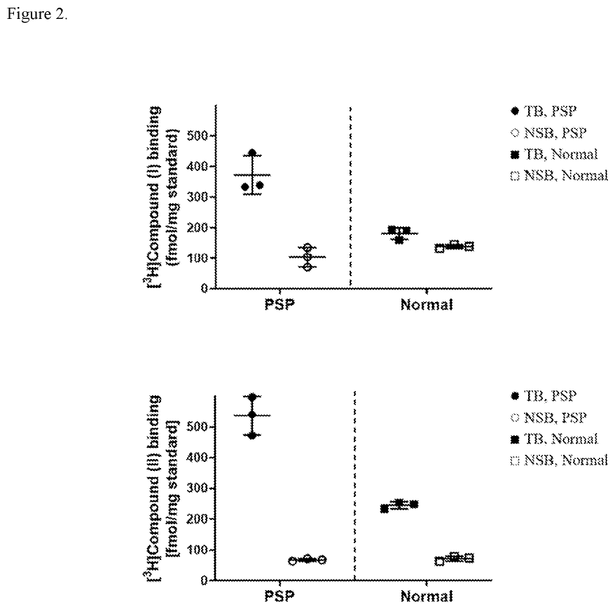

Quantitative analysis of the in vitro autoradiography results. Quantitative analysis was performed by computer assisted micro-densitometry (MCD; MCID basic, Interfocus, Devon, UK). MCD per mm² values were converted to the corresponding radioligand concentration (fmol/mg) by referring to tritium standards (American Radiolabeled Chemicals, Saint Louis, MO, USA) on the same plate. Identification of the areas of interest was determined by reference to the Atlas of the Human Brain (Mai et al., 2015, Elsevier, ISBN:9780128028018). Quantification of radioligand binding in normal and PSP brain was determined by selecting the whole grey matter area in the sections containing globus pallidus and putamen and by measuring the average binding intensity in the area selected (FIG. 2).

Determination of Compound (I) and Compound (II) affinity for PSP tissue by means of in vitro autoradiography. To measure the affinity of Compound (I) and Compound (II) for PSP brain tissue, homologous displacement (alias self-displacement) curves were obtained by incubating consecutive FF brain slices with 3 nM radioligand ([³H] Compound (I) or [³H] Compound (II)) alone, or in the presence of increasing concentrations (from 10¹⁰ to 10⁻⁴ M) of the corresponding non-radiolabeled ligand (Compound (I) or Compound (II), respectively). The concentrations inhibiting 50% of total binding (IC₅₀) was obtained by fitting the binding intensity data with the function “One site—Fit log IC50” of the software GraphPad Prism (GraphPad Software, San Diego, CA, USA). The constant of inhibition (Kᵢ) was calculated using the Cheng-Prusoff equation (Cheng and Prusoff, Biochem Pharmacol 1973 Dec. 1; 22(23):3099), considering the dissociation constant (K_{D}) of the radioligand numerically identical to the Kᵢ (i.e., by putting K_{D}=Kᵢ). To distinguish the affinity values obtained in such self-displacement experiments from the Kᵢ values obtained in heterologous displacement experiments and the K_{D} values obtained in radioligand saturation experiments, the affinity values obtained in self-displacement experiments are indicated as ^{sd}K_{D} (K_{D} determined by means of self-displacement experiments).

Binding of [³H] Compound (I) and [³H] Compound (II) binding to the globus pallidus and putamen of PSP and normal brain. [³H] Compound (I) and [³H] Compound (II), at 3 nM concentration, bound with an uneven distribution pattern to the FF tissue sections of globus pallidus and putamen from brains of PSP donors. These tissue sections were characterized by the presence of punctate binding in the globus pallidus and putamen of PSP brain, with much higher density in the globus pallidus. In contrast, binding was homogenous in the same regions of normal, age-matched donors and in the presence of 10 μM of the corresponding non-radiolabeled compound (FIG. 1). The mean binding density in the grey matter of globus pallidus and putamen was higher in the PSP brains than the normal brains (FIG. 2). At 3 nM radioligand concentration, the amount of [³H] Compound (I) bound to the tissue was 2.1 times higher in PSP brains than in normal brains. The amount of [³H] Compound (II) bound to the tissue following incubation with 3 nM radioligand concentration was 2.2 times higher in PSP brains than in normal brains (FIG. 2). The pattern of distribution of the radioligands binding was in line with the burden and distribution of 4R tau in PSP as reported by Williams (Brain, 2007,130, (6): 1566-1576). A similar distribution pattern, namely punctuate binding in PSP brain but not in normal brain, was also observed when using FFPE sections (FIG. 3).

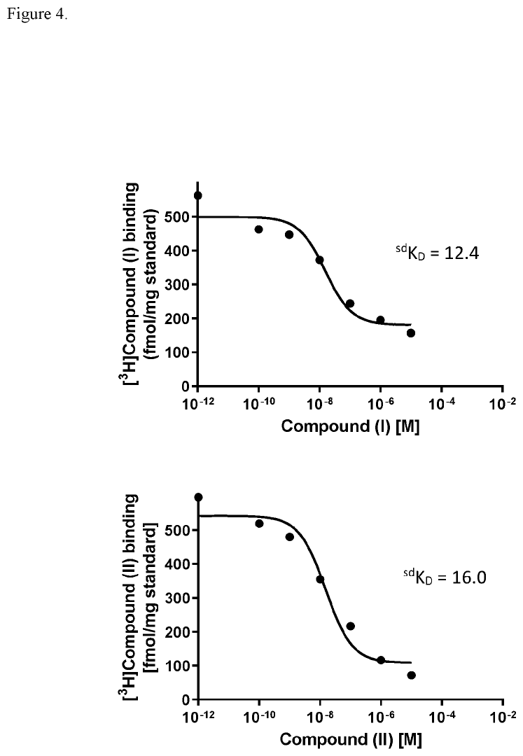

Affinity of Compound (I) and Compound (II) for tau aggregates in globus pallidus and putamen of PSP brains. Unlabeled Compound (I) and Compound (II) were able to displace with high potency [3H] Compound (I) and [³H] Compound (II), respectively, from PSP brain sections of globus pallidus and putamen (see FIG. 4). The ^{sd}K_{D} value calculated in these self-displacement experiments was 12.4 and 16.0 nM for [³H] Compound (I) and [³H] Compound (II), respectively (FIG. 4).

In summary, analysis of the in vitro data demonstrated several important properties of Compound (I) and Compound (II). First, both [³H] Compound (I) and [³H] Compound (II) showed a high degree of selectivity for tau aggregates in globus pallidus and putamen in PSP affected brain tissue as compared to healthy brain tissue. The total binding of [³H] Compound (I) and [³H] Compound (II) was significantly higher in the globus pallidus and putamen in PSP affected brain tissue, and could be titrated with unlabelled Compound (I) or Compound (II), respectively. The specificity of the interaction of these compounds to the PSP-affected brain tissue was shown by their ^{sd}K_{D} for Compound (I) at 12.4 nM, and at 16.0 nM for Compound (II). Overall, these data indicate that Compound (I) and Compound (II) were highly specific for binding to tau in the globus pallidus and putamen of PSP affected brain tissue.

### Example 8. Positron Emission Tomography (PET) Imaging and Interpretation

In the PET imaging studies, the following abbreviations were commonly used:

| | |
|---|---|
| SUV | Standard Uptake Value |
| TAC | Time Activity Curves |
| V_{T} | Total Volume of Distribution |

PET imaging procedures in cynomolgus macaques were performed under the guidelines of the Institutional Animal Care and Use Committee. Anesthesia was maintained using isoflurane gas maintained at 1.25-3.0% for the duration of the imaging procedures. Prior to PET imaging, a head computed tomography (CT) was acquired using a Neurologica CereTom® CT system (Neurologica, Danvers, MA, USA) which was used for localization of the animal in the PET system, and later for attenuation correction of the PET data.

The radiotracer was administered as a bolus injection (3 mL over 2 minutes), and data collected in dynamic list mode emission acquisition was initiated using a Siemens Concorde microPET® Focus™ 220 (Siemens USA, Washington DC, USA) for 120 minutes. Arterial blood samples were acquired throughout the PET acquisition to measure radioactivity in whole blood and plasma and to assess the % parent and metabolite(s) for generation of an input function for kinetic modeling analysis. The whole blood arterial samples were acquired, initially rapidly (every 30 seconds) and decreased in frequency towards the end of the PET acquisition to every 30 minutes. Arterial whole blood samples were counted in a well counter (PerkinElmer Wizard® 1470; PerkinElmer, Waltham, MA, USA) for radioactivity concentration. Samples were then centrifuged (3000 RCF for 5 minutes) to allow extraction of plasma which was subsequently counted for radioactivity concentration. To determine the proportion of unchanged radiotracer, selected plasma samples (1, 2, 3, 4, 7, 9, 15, 20, 40, 60, 90, and 120 minutes) were extracted with equal volumes of acetonitrile, then injected onto a Dionex/Thermo Ultimate 3000 radio-HPLC system (ThermoFisher, Waltham, MA, USA) consisting of a Phenomenex® Onyx™ C18 10×100 mm column (Torrance, CA, USA), an Eckert and Ziegler coincidence BGO radiodetector (model B-FC-4100, Eckert and Ziegler, Atlanta, GA, USA) and a mobile phase of (A) 10 mM ammonium acetate and (B) acetonitrile. The conditions for [¹⁸F] Compound (I) are: 0-2.5 min, 95% A at 4 mL/min; 2.5-2.6 min, 95% A to 50% A at 4 mL/min to 3 mL/min; 2.6-10 min, 50% A at 3 mL/min; 10-10.1 min, 50% A to 95% A at 3 mL/min to 4 mL/min; 10.1-13 min, 95% A at 4 mL/min. Retention time for [¹⁸F] Compound (I) was 8.4 min. The conditions for [¹⁸F] Compound (II) are: 0-2.5 min, 95% A at 4 mL/min; 2.5-2.6 min, 95% A to 65% A at 4 mL/min to 3 mL/min; 2.6-10 min, 65% A at 3 mL/min; 10-10.1 min, 65% A to 95% A at 3 mL/min to 4 mL/min; 10.1-13 min, 95% A at 4 mL/min. Retention time for [¹⁸F] Compound (II) was 9.3 min. Arterial sampling data was used to generate the input function for kinetic modeling. Potential off-target specific binding in the brain was evaluated for each lead radiotracer using a pre-dosing paradigm in which PET was performed after administration of a 1 mg/kg homologous dose of the non-radioactive tracer over a 15-minute infusion 20 minutes prior to radiotracer injection.

Upon the completion of PET image acquisition, anesthesia was terminated, primates were monitored until fully alert, then transported to their cages. In addition to PET imaging, each primate underwent separate MRI imaging (GE Discovery™ 3T MR750 MRI scanner and GE Signa™ HDxt 1.5T MRI scanner, GE Healthcare, Chicago, IL, USA) for acquisition of a structural T1w image for purposes of co-registration and transformation to a standard atlas space. The PET data analysis was performed, and images were reconstructed using Siemens MicroPET® Manager and AsiPro (Siemens) software. PET list mode data was binned into sinograms with time frames of 6×30 seconds, 3×1 minutes, 2×2 minutes, 22×5 minutes. Images were reconstructed using filtered back projection using a Hanning filter with a 0.5 cycles/pixel cutoff. Corrections for attenuation (using the acquired CT), scatter, and deadtime correction were applied. Furthermore, the dynamic images were decay-corrected to start of PET acquisition, which coincided with the radiotracer injection. Final reconstructed dynamic images had voxel sizes of 1.898×1.898×0.796 mm and a matrix size of 128×128×95 (x, y, z). Dynamic PET imaging data, structural CT and MRI images, and ancillary data (arterial blood data, injected radiotracer information, subject information) were processed resulting in dynamic PET images in atlas space allowing for regional and voxelwise assessment of radiotracer kinetics. In short, to allow co-registration, non-attenuation corrected PET image reconstructed from the whole scanning duration was rigidly aligned to the subject's head CT image which was then rigidly aligned to the subject's structural MRI image. The MRI dataset was then transformed into the standard space using linear and nonlinear warping methods. Aligning to a standard space allowed within and between subject comparison and extraction of time activity curves (TACs) using atlas-based regions of interest (ROIs). An MRI based subject specific gray matter segmentation was applied to the atlas to exclude parts of the ROTs outside of what was considered gray matter for that particular subject. ROIs examined in this work included the gray matter, cerebral white matter, cerebellum, frontal lobe, parietal lobe, temporal lobe, occipital, globus pallidus and putamen. Regional TACs were fit using standard one- (1T) and two-tissue reversible (2T) and irreversible (2Ti) compartmental models for estimation of kinetic parameters including total volume of distribution (V_{T}). All compartmental configurations were assessed using both a fixed cerebral vascular fraction of 5% and as a fitted model parameter (v). Effectiveness of compartmental configurations were assessed using the Akaike Information Criterion (AIC) which penalizes models for increased complexity and poor fits. All co-registrations and kinetic modeling were performed with MIAKAT™ (Invicro, Konica-Minolta, Boston, MA, USA) which runs within the MATLAB® environment (MATLAB 2019b, MathWorks, Natick, MA, USA). Reference region analysis was performed using the cerebellar gray matter as a reference region to allow comparison of V_{T} and blood-based distribution volume ratio (DVR) under baseline and homologous block conditions.

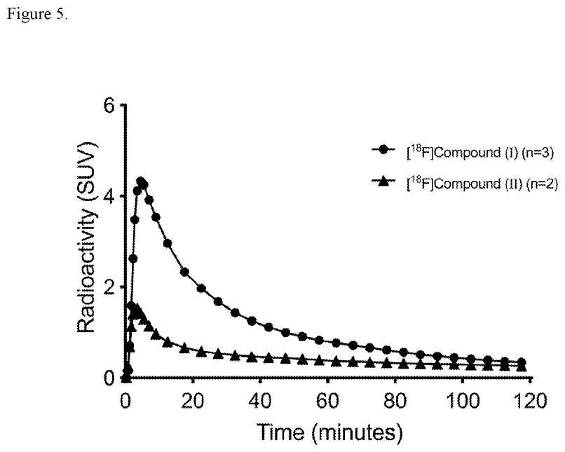

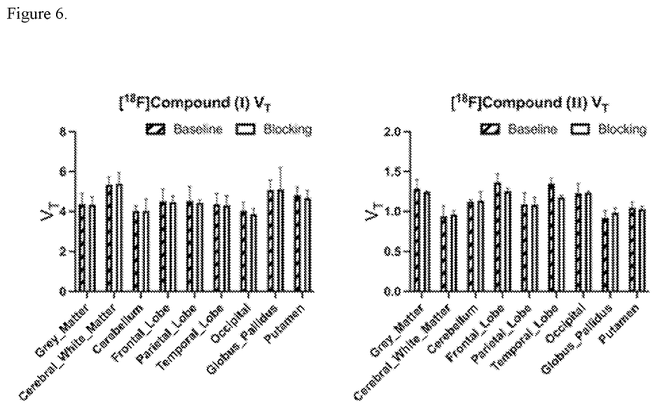

The results of the PET imaging, shown in FIG. 5, are time activity curves (TACs) extracted from gray matter with compartmental modeling fits using the two-tissue compartment model which was the preferred model by Akaike information criterion (AIC). FIG. 6 highlights the regional V_{T} values during baseline and following self-blocking administration of the corresponding non-radiolabeled compound. After injection of radiotracer, brain radioactivity concentrations were initially high (SUVs ~2-4, with [¹⁸F] Compound (I) displaying the highest uptake) and rapidly decreased after the peak in brain uptake at approximately 5 minutes. Toward the end of the PET measurements, no significant differences in regional uptake were observed. The distribution of radioactivity was relatively homogenous across the examined brain regions and is consistent with an absence of target-specific binding of either tracer in these healthy nonhuman primates. In addition, no statistical difference in the V_{T} values between the baseline and self-block experiments indicate little to no off-target binding of these tracers.

In summary, the PET imaging studies with cynomolgus macaques demonstrated several significant findings related to Compound (I) and Compound (II). First, both [¹⁸F] Compound (I) and [¹⁸F] Compound (II) showed the ability to cross the blood brain barrier and were characterized by rapid uptake and rapid clearance. Although healthy cynomolgus macaques do not show 4R tauopathies, the loading of the [¹⁸F] Compound (I) and [¹⁸F] Compound (II) was uniform across the brain and did not show significant off-target binding.

What is claimed:

1. A compound of Formula (I), or a pharmaceutically acceptable salt thereof: 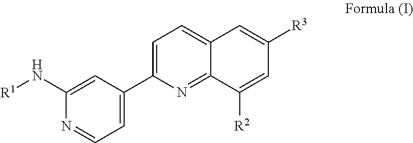
wherein:
R¹ is CH₃ or C(³H)₃;
R² is H, F or ¹⁸F;
R³ is —O—CH₂CH₂—O—CH₃, or 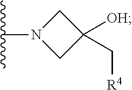
and
R⁴ is F or ¹⁸F.

2. The compound of claim 1, or a pharmaceutically acceptable salt thereof, wherein:
R¹ is CH₃ or C(³H)₃;
R² is F or ¹⁸F; and
R³ is —O—CH₂CH₂—O—CH₃.

3. The compound of claim 2, or a pharmaceutically acceptable salt thereof, wherein:
R¹ is CH₃; and
R² is F.

4. The compound of claim 2, or a pharmaceutically acceptable salt thereof, wherein:
R¹ is CH₃; and
R² is ¹⁸F.

5. The compound of claim 2, or a pharmaceutically acceptable salt thereof, wherein:
R¹ is C(³H)₃; and
R² is F.

6. The compound of claim 1, or a pharmaceutically acceptable salt thereof, wherein:
R¹ is CH₃ or C(³H)₃;
R² is H;
R³ is 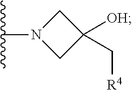
and
R⁴ is F or ¹⁸F.

7. The compound of claim 6, or a pharmaceutically acceptable salt thereof, wherein:
R¹ is CH₃; and
R⁴ is F.

8. The compound of claim 6, or a pharmaceutically acceptable salt thereof, wherein:
R¹ is CH₃; and
R⁴ is ¹⁸F.

9. The compound of claim 6, or a pharmaceutically acceptable salt thereof, wherein
R¹ is C(³H)₃; and
R⁴ is F.

10. The compound of claim 1, or a pharmaceutically acceptable salt thereof, selected from the group consisting of:
4-[8-fluoro-6-(2-methoxyethoxy)quinoline-2-yl]-N-methylpyridin-2-amine;
4-[8-(¹⁸F)fluoro-6-(2-methoxyethoxy)quinoline-2-yl]-N-methylpyridin-2-amine;
4-[8-fluoro-6-(2-methoxyethoxy)quinolin-2-yl]-N—(³H₃)methylpyridin-2-amine;
3-(fluoromethyl)-1-{2-[2-(methylamino)pyridin-4-yl]quinolin-6-yl}azetidin-3-ol;
3-[(¹⁸F)fluoromethyl]-1-{2-[2-(methylamino)pyridin-4-yl]quinolin-6-yl}azetidin-3-ol; and
3-(fluoromethyl)-1-(2-{2-[(³H₃)methylamino]pyridin-4-yl}quinolin-6-yl)azetidin-3-ol.

11. The compound of claim 1, wherein the compound is 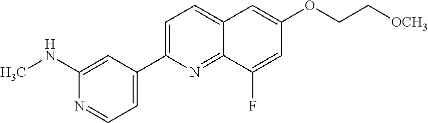 [SMILES: CNc1cc(-c2ccc3cc(OCCOC)cc(F)c3n2)ccn1]
or a pharmaceutically acceptable salt thereof.

12. The compound of claim 1, wherein the compound is

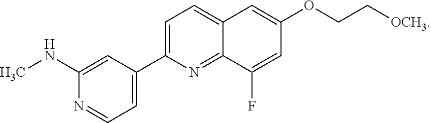 [SMILES: CNc1cc(-c2ccc3cc(OCCOC)cc(F)c3n2)ccn1]

13. The compound of claim 1, wherein the compound is 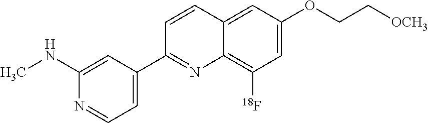 [SMILES: CNc1cc(-c2ccc3cc(OCCOC)cc([18F])c3n2)ccn1]
or a pharmaceutically acceptable salt thereof.

14. The compound of claim 1, wherein the compound is

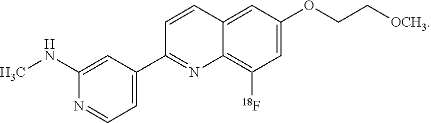 [SMILES: CNc1cc(-c2ccc3cc(OCCOC)cc([18F])c3n2)ccn1]

15. The compound of claim 1, wherein the compound is 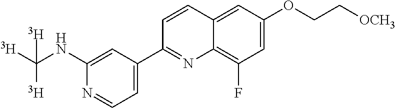 [SMILES: [3H]C([3H])([3H])Nc1cc(-c2ccc3cc(OCCOC)cc(F)c3n2)ccn1]
or a pharmaceutically acceptable salt thereof.

16. The compound of claim 1, wherein the compound is

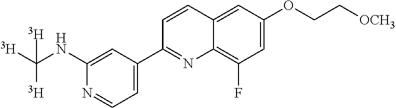 [SMILES: [3H]C([3H])([3H])Nc1cc(-c2ccc3cc(OCCOC)cc(F)c3n2)ccn1]

17. The compound of claim 1, wherein the compound is 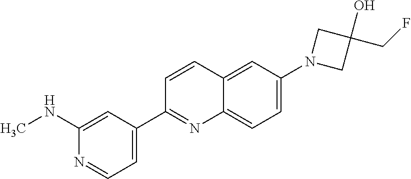 [SMILES: CNc1cc(-c2ccc3cc(N4CC(O)(CF)C4)ccc3n2)ccn1]
or a pharmaceutically acceptable salt thereof.

18. The compound of claim 1, wherein the compound is

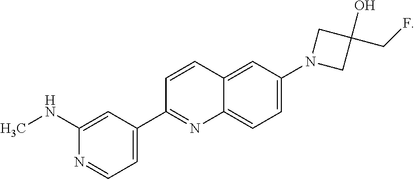 [SMILES: CNc1cc(-c2ccc3cc(N4CC(O)(CF)C4)ccc3n2)ccn1]

19. The compound of claim 1, wherein the compound is 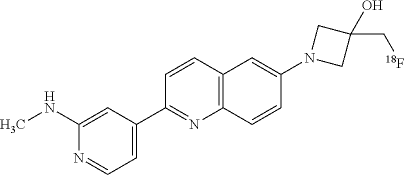 [SMILES: CNc1cc(-c2ccc3cc(N4CC(O)(C[18F])C4)ccc3n2)ccn1]
or a pharmaceutically acceptable salt thereof.

20. The compound of claim 1, wherein the compound is

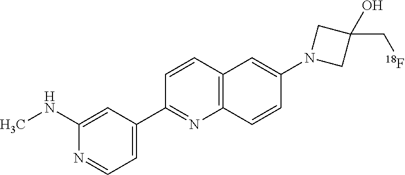 [SMILES: CNc1cc(-c2ccc3cc(N4CC(O)(C[18F])C4)ccc3n2)ccn1]

21. The compound of claim 1, wherein the compound is 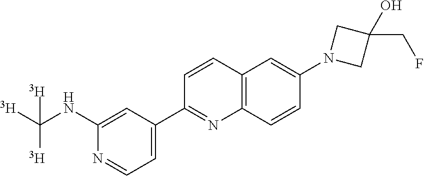 [SMILES: [3H]C([3H])([3H])Nc1cc(-c2ccc3cc(N4CC(O)(CF)C4)ccc3n2)ccn1]
or a pharmaceutically acceptable salt thereof.

22. The compound of claim 1, wherein the compound is

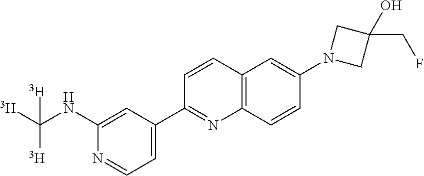 [SMILES: [3H]C([3H])([3H])Nc1cc(-c2ccc3cc(N4CC(O)(CF)C4)ccc3n2)ccn1]

23. A pharmaceutical composition comprising a therapeutically or diagnostically effective amount of a compound of Formula (I) according to claim 1, or a pharmaceutically acceptable salt thereof, in combination with a pharmaceutically acceptable carrier.

24. A method of imaging tau aggregates in a subject, the method comprising administering a diagnostically effective amount of a radiolabeled compound according to claim 1 to a patient and imaging tau aggregates in the patient.

25. The method of claim 24, wherein the radiolabeled compound is 4-[8-(¹⁸F)fluoro-6-(2-methoxyethoxy)quinoline-2-yl]-N-methylpyridin-2-amine.

26. The method of claim 24, wherein the radiolabeled compound is 3-[(¹⁸F)fluoromethyl]-1-{2-[2-(methylamino)pyridin-4-yl]quinolin-6-yl}azetidin-3-ol.

27. The method of claim 24, wherein the subject is experiencing or is suspected to be experiencing a tauopathy.

28. The method according to claim 27, wherein the tauopathy is progressive supranuclear palsy.

29. The method according to claim 24, wherein the tau aggregates are 4R tau aggregates.
